(19) United States
(12) Patent Application Publication
(10) Pub. No.: US 2004/0189718 A1
(43) Pub. Date: Sep. 30, 2004

(54) Medic-to-medic/map of medicine

(75) Inventors: Michael Lewis Stein, Oxford (GB); Owen Epstein, London (GB)

Correspondence Address: OPPENHEIMER WOLFF & DONNELLY LLP, 45 SOUTH SEVENTH STREET, SUITE 3300, MINNEAPOLIS, MN, 55402, US

(73) Assignee: MEDIC-TO-MEDIC LIMITED, London (GB)

(21) Appl. No.: 10/808,142

(22) Filed: Mar. 24, 2004

(30) Foreign Application Priority Data
Mar. 24, 2003 (GB) 0306746.9

(51) Int. Cl. G09G 5/00

(52) U.S. Cl. 345/853

(57) ABSTRACT

A graphical user interface (GUI) for interacting with a user during a workflow process is described. The GUI comprises a page including a plurality of interlinked nodes which graphically represent the structure of a plurality of interlinked steps of a stored workflow process; data entry means for entering data relating to a particular selected node; wherein the node has a unique relationship with a step in the workflow process; pathway means for determining a particular path through the workflow process using the entered data; and means for graphically representing the resultant path through the workflow process in the page.

### FIELD OF THE INVENTION

The present invention concerns improvements relating to graphical user interfaces and provides, more specifically, a graphical user interface (GUI) for use by health professionals in diagnosing and treating patients.

### BACKGROUND TO THE INVENTION

Healthcare providers are under continual pressure to cut costs whilst improving standards of care and they are increasingly looking to information technology to help them meet these challenges. By reducing the time spent by staff on certain tasks, healthcare organisations can save on labour costs, treat more patients and become more efficient. One of the biggest obstacles to increased efficiency are legacy paper-based systems, in particular those concerning patient records.

Patient records are a legal requirement and are used by health professionals to record a patient's health history. The majority of patient records still comprise information collated on paper, with separate sets of records being kept by different healthcare institutions such as hospitals, general practitioner surgeries etc. Indeed, even within the same hospital, a patient may have a number of records held separately by whichever consultant, clinic or ward from which they are receiving treatment. The physical management of such records places a significant burden on the healthcare provider—for example, a large district general hospital needs to store between 40,000 and 80,000 patient records on-site per year. Whilst less frequently accessed records can be archived off-site, the on-site storage facilities take up valuable space which could otherwise be used to accommodate further treatment areas, hospital beds, office space for administrative staff or even car parking spaces. The financial costs associated with managing paper-based patient records, namely filing, retrieving and delivering the records to their required destination, are significant. The records cannot be quickly transferred around healthcare systems and it is also not uncommon for patient records to be mislaid or lost, which can lead to delays in patient treatment (with the patient having to be sent home, in certain circumstances, after arriving for a scheduled appointment) and concerns for patient confidentiality.

In addition, extracting any kind of information from paper records in order to perform analysis is cumbersome and expensive. To this end, local Patient Administration Systems (PAS) have been introduced in hospitals to provide a high-level electronic summary of the paper-based records for administrative purposes. The information on a PAS record may comprise patient number, date of birth, dates of admission, treatment and discharge, the name of the consultant under whom the patient is receiving treatment and codes indicating diagnoses and procedures (namely under the International Classification Codes of Diseases, ICD, and the Operating Procedure Codes System, OPCS, respectively). Information can then be readily extracted from the PAS records to generate statistical information on the patient care provided by the hospital, either for internal use or as feeds into wider demographic review systems. The systems can also additionally help in the tracking of patient records. However, the key function is an administrative one rather than clinical—the PAS records do not contain the detailed information required by health professionals to treat patients.

The difficulties and problems associated with paper-based patient record systems are set to become exacerbated over the coming years, as the ageing population places increasing demands on healthcare systems, with ever-greater numbers of patients requiring treatment at any one time. Efficient patient records management is viewed as being fundamental to the future delivering of quality patient care and it is hoped that this can be realised through the introduction of electronic patient records (EPR), putting an end to the paper-chasing practices of the past. EPR systems have already been successfully implemented in trials, bringing all records for a single patient in one institution together in an electronic format which is accessible from any workstation that is networked to the institution's electronic patient record management system (EPRMS).

EPRMSs are localised at present, with each hospital/general practitioner's surgery implementing separate systems. However, nationalised systems are seen as the way forward, so that information about patients will be mobile like the patients themselves and be readily available to authorised healthcare professionals wherever the patient requires care.

Furthermore, it is intended to develop the EPRMs to provide healthcare professionals with a full suite of software applications which will enable them to view, process and complete patient records on a single workstation, without additionally having to use manual or other automated systems.

One key concern is keeping medical professionals up-to-date with new research and best practice guidelines on how to diagnose and treat conditions. At present medical best practice guidelines are arrived at by conducting a review of published research literature, going through a consensus process and evaluating any available evidence, the guidelines being subject to review and approval by peers. The guidelines are then published and disseminated amongst practitioners. However, there is then a reliance on healthcare professionals reading and internalising the guidelines, employing them in practice as and when appropriate circumstances arise. In reality, this presents practitioners with an onerous task and many struggle to keep fully abreast of medical developments in the face of their demanding workloads, particularly general practitioners.

There are also huge time pressures to be contended with in the consulting environment, both within the specialist hospital consulting environment and general practitioner surgeries. For example, in the UK the average consultation time in a general practitioner's surgery is between 8 and 10 minutes. During this time the general practitioner has to review the patient's record, interview the patient, perform any necessary examinations, diagnose, select an appropriate form of treatment and issue a prescription.

In certain situations, the health professional may need to find out more information about a particular symptom or condition. Whilst the Internet has made a wealth of material available through desktop terminals, searches of the World Wide Web typically return tens of results which need to be assessed and discounted until the relevant information is obtained, all of which takes valuable time that is not available in the consultation environment. In addition, information published on the Internet is difficult to regulate and not subject to the same rigorous assessment as peer-reviewed healthcare literature. Nevertheless, the demands on healthcare professionals to deliver care are so great that, at times, they can be pressurised into relying on such information.

Regulated information is more likely to be available through proprietary third party knowledge bases. However, such resources can be cumbersome and time-consuming to use. Any one system is unlikely to satisfy all of a practitioner's needs, such that different systems require the practitioner to know and use different use skills in order to elicit the desired information.

Attempts to make guidelines available to practitioners at the point of care through EPRMs have been explored by the GLIF and SAGE research projects amongst others. The GLIF project has developed a common language for representing clinical guidelines, namely the so-called GuideLine Interchange Format, and its goal is to make the GLIF representations available to healthcare organisations so that the guidelines can be adapted for use with local clinical information systems (it is recognised that healthcare institutions are unlikely to accept generic guidelines without at least some minor local modifications). In contrast, the Standards-based sharable Active Guideline Environment (SAGE) has concentrated its efforts on how to best integrate guideline-based decision-support systems with local clinical information systems. The SAGE decision support engine integrates guideline recommendations, as well as access to evidence and rationale, into existing clinical workflows. However, clinical support systems implemented to date using either the GLIF and SAGE methodologies require the healthcare professional to make a preliminary diagnosis—they then provide information which supports the diagnosis and advise of any additional checks or actions which are required.

In reality, diagnosis in the consulting environment is not always straightforward. Patients typically present one or more new symptoms which must be considered in conjunction with existing conditions and previous medical history. GLIF and SAGE rely on the existence of pre-coded care pathways within the clinical support system—if the newly presented symptom has not been considered in combination with the existing diagnosis and other details of the patient's history, then an appropriate care pathway will not exist. Hence, the inherent rigidness of these methodologies prevents them from meeting the needs of healthcare professionals at the point of care, since it is impossible to create predetermined pathways for every variation and combination that a patient may present.

It is desired to overcome or substantially reduce some of the abovementioned problems. More specifically, it is desired to provide a graphical user interface which users in carrying out their function, for example assisting healthcare professionals in the delivery of patient care, to promote faster interaction with the supporting information which can be provided via the graphical user interface. This, in the context of healthcare professionals, enables them to provide care in accordance with best practice guidelines.

### SUMMARY OF THE INVENTION

The present invention resides in the appreciation that providing a graphical representation of steps of a workflow process can be highly advantageous when it comes to use of that interface for data entry and direction along a workflow process.

According to one aspect of the present invention there is provided a graphical user interface (GUI) for interacting with a user during a workflow process, the GUI comprising: a page including a plurality of interlinked nodes which graphically represent the structure of a plurality of interlinked steps of a stored workflow process; data entry means for entering data relating to a particular selected node; wherein the node has a unique relationship with a step in the workflow process; pathway means for determining a particular path through the workflow process using the entered data; and means for graphically representing the resultant path through the workflow process in the page.

The page of interlinked nodes provides a map of at least part of the workflow process to be traversed. The representation of the workflow in this manner allows simple and intuitive interaction with the user. As each node has a direct relationship to a step in the workflow process, data entry and user interaction is speeded up.

Preferably the plurality of interlinked nodes represent a complete workflow process on a single page. This is particularly advantageous as the user is immediately able to see their history as they traverse the different steps of the work flow as well as being able to determine the end point of a particular workflow at a glance.

The present invention has a significant advantage that in a multistage process, a plurality of stages are shown on a single page such that a user can see his history at a glance as well as being able to track back to where in the decision process he may have made a mistake in his diagnosis.

In an embodiment of the present invention useful information can be contextualised within a clinical best practice workflow and that a more effective graphical user interface can be achieved by enabling a healthcare practitioner to navigate through the workflow intuitively, starting from either a patient concern, suspected diagnosis or an exhibited symptom.

The data entry means preferable comprises presentation means for presenting data relevant to a location of the selected node within the plurality of interlinked nodes and selection means for enabling user selection of at least some of that data. In this way, a user can be presented with relevant data to enter depending on their position within the map and can select it simply. This advantageously speeds up and makes data entry easier for the healthcare practitioner for example.

Preferable the data entry means is arranged to use the entered data at a first node to determine further information required at a second node, linked to the first node. In this way specific data need only be entered once but can be used at different nodes many times.

The GUI may also comprise means for converting the entered data into a classification code representing that data. This enable a uniform representation of any data within the GUI to be achieved. This is particularly advantageous when linking to external systems where the same classification codes can be understood.

Preferably the GUI further comprises analysing means for analysing the entered data and generating a list of actions associated therewith and listing means for listing the list of associated actions to the user adjacent the plurality of displayed interlinked nodes. This enables tasks to be generated almost as a by-product of the process of navigating the map such that users are not only correctly guided by the underlying workflow but also have the benefit of having many of the actions they need to carry out as a consequence being determined. This list of actions can then be processed to automatically order such actions to occur. For example, a blood test could be ordered for a patient from the list.

The action list means may be arranged, at the end of traversal of a plurality of interlinked nodes comprising the page, to present the list to the user with options for user confirmation of each action, and to determine the list of actions to be implemented from the user confirmation. In this way only those actions which the user feels are required are carried out.

Each node preferably further comprises an information means provided at a node for presenting information associated with a node upon user selection. This helps the user to progress through the workflow and obtain any further relevant information required for decisions at each node.

The map is preferably customisable to accommodate user preferences. More specifically the GUI may further comprise a note recordal means for recording user-generated textual note relating to a particular node, the note recordal means being arranged to link the note with the particular node such that the stored note is retrievable when the user has navigated to that particular node.

The GUI may further comprise feedback generation means for converting a user-determined note into a transmittable message and for transmitting the message to another user having access to a version of the GUI. This enables questions arising from use of the map to be handled in a quick an effected manner and often assists in conveying the context of the feedback more accurately.

Preferably the GUI has access to an Electronic Patient Record Management System (EPRMS) and the GUI further comprises an EPRMS management means for obtaining and presenting details of a selected electronic patent record in a portion of the page. This is a highly advantageous aspect of the present invention. Integration with an electronic patient record can be highly beneficial in that previously stored information about the patient can be used to assist in the progression of the workflow. Furthermore, data obtained in the workflow process can be used to update a patient record at the same time thereby providing a more accurate view of the patient's history at all times.

The EPRMS management means may be arranged to use the details of the selected electronic patient record to determine what information is required at a node from the user. In this way the map is responsive to and can be shaped by the data already in the patient record.

The GUI preferably further comprises searching means for searching an externally accessible knowledge base, the searching means being arranged to convert a selected information topic into a predetermined classification code representing that topic and to transmit that classification code within an information request to the knowledge base for relevant information contained therein. The use of codes in this manner is highly advantageous as it enables direct access to knowledge bases without the requirement for a search to be carried out. This in turn minimises the time it takes to obtain the required information. In this regard preferably the classification code comprises a standard classification code describing a complete range of possible data inputs relevant to the subject of the workflow process. This facilitates improved coverage of requests and better compatibility.

The searching means may be arranged to receive a response to the information request and use the response to determine a relevant page of a plurality of pages for display to the user. Thus the information received can be used to direct the user to a specific starting point in the workflow process that is highly relevant to their search query.

Preferably the GUI further comprises editing means for editing the plurality of interconnected nodes on a page, the editing means being arranged to update the stored workflow to reflect any change made to the page. The editable nature of the GUI enables the user advantageously to account for any local variation in the workflow that is required. A level of authority for the user can determine the extent to which they are permitted to make changes to the map.

The GUI preferably further comprises recording means for recording user navigation through the plurality of interlinked nodes. This provides the user with a history of the path taken through the workflow which can be used in a number of ways.

The GUI may further comprise navigation analysis means for analysing the user navigation to determine the precise path taken through the workflow process. This navigation history can be used for auditing and for analysis of user performance.

According to another aspect of the present invention there is provided a graphical user interface (GUI) for interacting with a user during a workflow process, the GUI comprising: a map comprising a plurality of interlinked nodes which graphically represent the structure of a plurality of interlinked steps of a stored workflow process; a data entry module for entering data relating to a particular selected node; wherein the node has a unique relationship with a step in the workflow process; a pathway module for determining a particular path through the workflow process using the entered data; and a display module for graphically representing the resultant path through the workflow process in the map.

According to another aspect of the present invention there is provided a graphical user interface (GUI) for providing a user interface to a knowledge base storing a workflow process, the GUI comprising: a page including a plurality of interlinked nodes which graphically represent the structure of a plurality of interlinked steps of the stored workflow process; means for entering data relating to a particular selected node; wherein the node has a unique relationship with a step in the stored workflow process; means for determining a particular path through the workflow process using the entered data; and means for graphically representing the resultant path through the workflow process in the page.

According to another aspect of the present invention there is provided a graphical user interface (GUI) for interacting with a user during a workflow process, the GUI comprising: a plurality of pages representing a plurality of interrelated workflow processes, each page comprising a plurality of interlinked nodes which graphically represent the structure of a plurality of interlinked steps within a stored workflow process; data entry means for entering data relating to a particular selected node; wherein the node has a unique relationship with a step in the workflow process; determining means for determining a particular path through the workflow process using the entered data; and graphical means for graphically representing the resultant path through the workflow process in the page.

The present invention also extends to a method of interacting with a user during a workflow process using a graphical user interface (GUI), the method comprising: generating a page of the GUI, the page comprising a plurality of interlinked nodes which graphically represent the structure of a plurality of interlinked steps of the workflow process; entering data relating to a particular selected node; the node having a unique relationship with a step in the workflow process; determining a particular path through the workflow process using the entered data; and graphically representing the resultant path through the workflow process in the page.

According to another aspect of the present invention there is provided a graphical user interface (GUI) for interacting with a user during a workflow process, the GUI comprising: searching means for searching an externally accessible knowledge base, the searching means comprising: conversion means for converting a selected information topic into a predetermined classification code representing that topic; and transmission means for transmitting that classification code within an information request over a communications network to the knowledge base to access relevant information contained therein.

This GUI provides access to external knowledge bases without requiring any search to be carried out. This is highly advantageous as it enables faster more accurate access to the data contained within those knowledge bases.

A practical implementation is realised when the conversion means further comprises a local database of predetermined classification codes and an associated list of specific information topics which are each mapped to a specific classification code. Therefore using the local database, a topic specified by the user can be used to look up either previously or in real time the appropriate code for an information request.

The present invention also extends to a method of interacting with a user during a workflow process using a graphical user interface (GUI), the method comprising: receiving a user instruction from the GUI to search an externally accessible knowledge base; initiating a search of the knowledge base by: converting a selected information topic into a predetermined classification code representing that topic; and transmitting that classification code within an information request over a communications network to the knowledge base to access relevant information contained therein.

According to another aspect of the present invention there is provided a graphical user interface (GUI) for interacting with a user during a workflow process, the GUI comprising: a page including a plurality of interlinked nodes which graphically represent the structure of a plurality of interlinked steps of a stored workflow process; editing means for editing the plurality of interlinked nodes; and updating means for updating the plurality of interlinked steps of the stored workflow process with any corresponding changes made to the plurality of interlinked nodes. Enabling editing of a workflow representation in this way is highly advantageous as it enables local variation of the workflow representation to be carried out. This mitigates the inflexibility of the prior art systems described previously.

According to another aspect of the present invention there is provided a system for supporting distributed interaction with a user during a workflow process, the system comprising: a centrally stored graphical representation of the workflow process; a plurality of users located remotely from the centrally stored representation and related to each other in a user hierarchy, each user having access to a version of the representation; referral means provided within each version of the representation to generate a referral message, the referral means being arranged to send the message to a reviewer in a next higher level in the user hierarchy.

This aspect of the present invention enables feedback to be generated and dealt in a controlled manner by a system in which there may be hundreds of thousands of users.

According to another aspect of the present invention there is provided a system for distributing a new version of a graphical user interface (GUI) to a user, the system comprising: a central store retaining a GUI representation of a workflow process; a plurality of users located remotely from the central store and related to each other in a user hierarchy below the central store, each user having access to a version of a previous representation; comparing means for comparing the new version of the representation with a user's previous version of the representation to determine any differences; forwarding means for forwarding those differences to the user associated with that version of the representation for consideration; and reviewing means provided within each previous version of the representation, the reviewing means being arranged to accept or reject the differences and to convey an acceptance or rejection to a higher level within the hierarchy.

This provides a way of distributing updates amongst many users in a controlled manner which enables content editors the ability to control what is accepted.

According to another aspect of the present invention there is provided a method of constructing a graphical user interface, the method comprising: collating content regarding a particular workflow; recording that content in a database as a series of steps of a hierarchically structured workflow; and generating a graphical representation of the hierarchical workflow structure, which can be used to guide a user through the workflow; the graphical representation comprising a plurality of interlinked nodes where each node corresponds to a specific point within the hierarchical workflow structure.

This is one novel aspect of the present invention, namely that the software application and GUI are designed and built around the content of the Map, facilitating the later addition of software applications to the content. All other applications in this arena have been built as software applications first and foremost and add content later.

### BRIEF DESCRIPTION OF THE DRAWINGS

Methods and apparatus according to preferred embodiments of the invention for delivering improved patient care via a graphical user interface will now be described by way of example, with reference to the accompanying drawings in which:

FIG. 1 is a schematic diagram showing a communications system for providing the graphical user interface from a proprietary system, comprising a server and database, to various institutions within a healthcare system, according to exemplary embodiments of the present invention;

FIG. 2 is a schematic block diagram showing the software modules incorporated in the proprietary server of FIG. 1, including an Editing Tool Application, comprising Clinical and Admin Modules, for use in creating and editing patient care pathways displayed by the graphical user interface;

FIG. 3 is a schematic diagram showing the contents of the proprietary database of FIG. 1;

FIGS. 4a to 4f are a series of screen shots according to a first embodiment of the graphical user interface, showing the interface functionality when integrated with an EPRMS;

FIGS. 5a to 5d are a series of screen shots according to a second embodiment of the graphical user interface, when the interface is not integrated with an EPRMS, showing searching functionality;

FIGS. 6a and 6b are a series of screen shots according to a third embodiment of the graphical user interface, again when the interface is not integrated with an EPRMS, showing audit functionality;

FIGS. 7a to 7e are a series of screen shots showing a patient care pathway, for display by the graphical user interface, being extended using the Editing Tool Application of FIG. 2;

FIGS. 8a to 8e are a series of screen shots showing the incorporation of clinical and administration data within the patient care pathway of FIGS. 7a to 7e using the Clinical and Admin Modules of FIG. 2;

FIG. 9 is a schematic diagram showing the distribution of new versions of the graphical user interface down through the hierarchical levels within the healthcare system; and

FIG. 10 is a schematic diagram showing the distribution of feedback, relating to a patient care pathway and initiated through the graphical user interface, up through the hierarchical levels within the healthcare system.

### DETAILED DESCRIPTION OF PRESENTLY EMBODIMENTS OF THE PRESENT INVENTION

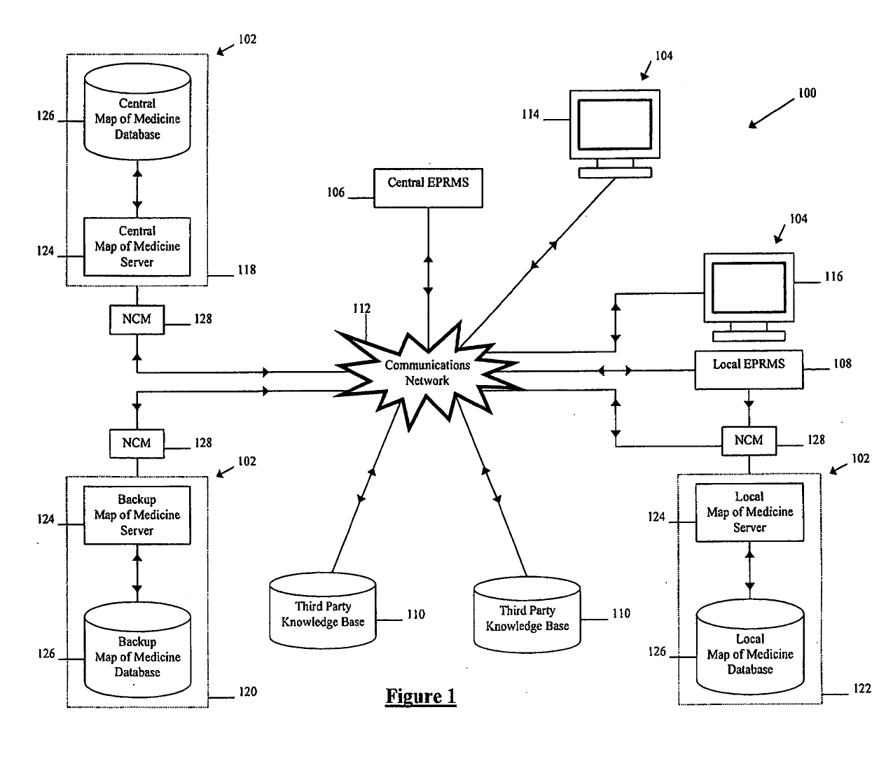

With reference to FIG. 1, a communications system 100 for implementing presently preferred embodiments of the present invention will presently be described. The communications system 100 facilitates communications between healthcare institutions and a proprietary system providing a graphical user interface through which a portfolio of electronic diagnosis and treatment tools is available to assist in the delivery of patient care. The graphical user interface presents a series of patient care pathways, commencing with either a suspected diagnosis or exhibited symptom, in the form of a graphical representation of a clinical workflow or a roadmap. Each patient care pathway conforms with best practice guidelines and is broken down into a series of action or decision points which are hereinafter referred to as nodes. All of the necessary information and software tools that a healthcare practitioner requires to properly manage a patient are embedded in the roadmap, hereinafter referred to as the Map of Medicine, at the appropriate node. The interface is able to both guide and record the route traversed by the healthcare practitioner across the Map. The guidance function helps to optimise patient care in accordance with clinical guidelines, whilst the recording functionality additionally allows the Map of Medicine to be used for training and audit purposes.

The communications system 100 mentioned above is comprised of a distributed proprietary system 102, a plurality of computing devices 104 located in various healthcare institutions, a Central EPRMS 106 and any Local EPRMSs 108 accessed by the healthcare institutions (whose data are periodically uploaded to the Central EPRMS 106), a plurality of Third Party Knowledge Bases 110 and a Communications Network 112, to which all of the above are connected. The Map of Medicine is provided to computing devices 104 within the healthcare institutions, via the Communications Network 112, by the proprietary system 102. The Communications Network 112 is an open network having secure aspects that are virtually closed although physically linked—accordingly it can be thought of as a virtual private network within a general wide area communications network (not shown) such as the Internet. Information from the Third Party Knowledge Bases 110 is accessible to the computing devices 104 through nodes on the Map of Medicine interface.

In FIG. 1, only two computing devices 104, one Local EPRMS 108 and two Third Party Knowledge Bases 110 are shown. Both computing devices 104 are taken to be computing terminals for the purposes of the present description: a first computing terminal 114 accesses the Central EPRMS 106 and a second computing terminal 116, located in a different healthcare institution from the first, accesses the Local EPRMS 108. Information from electronic patient records stored by the Central EPRMS 106 can be incorporated into the Map of Medicine as provided to the first computing device 114, whilst information from electronic patient records stored by the Local EPRMS 108 can be incorporated into the Map of Medicine as provided to the second computing device 116. Alternatively, each of the computing terminals 114 and 116 can access the Map of Medicine independently of their respective EPRMSs, for example by typing in a specific Uniform Resource Identifier (URL) into a browser (not shown) of the computing device 104.

The distributed proprietary system 102 is comprised of a central proprietary sub-system 118, a backup proprietary sub-system 120 and a plurality of local proprietary sub-systems 122 (of which only one is shown in FIG. 1). Each of the proprietary sub-systems 116, 118 and 120 comprises a Map of Medicine Server 124 and a Map of Medicine Database 126 and connects to the Communications Network 112 via a Network Communications Manager (NCM) 128. In the case of the local proprietary sub-system 122, the NCM 128 also connects the sub-system 122 to the local EPRMS 108.

The central proprietary sub-system 118 stores and provides both a master copy of the Map of Medicine and localised versions of the Map of Medicine, as well as data associated with the same. Localised versions of the Map are those which have been customised by local healthcare institutions for use by their clinical staff, for example specifying one particular drug for treatment over another because of cost issues. The central proprietary sub-system 118 is replicated by the backup proprietary sub-system 120, such that the Map of Medicine can still be provided to healthcare institutions even in the event of central systems failure. In addition, certain local healthcare institutions (or groups of institutions) may be of a sufficient size to warrant having their localised version of the Map of Medicine, and associated data, stored and provided by a local proprietary sub-system 122, although copies of the localised Maps and the associated data would still additionally be maintained by the central proprietary sub-system 118 and the backup proprietary sub-system 120. The key benefit of having local proprietary sub-systems 122 to handle the Maps of Medicine in a particular geographic area is the reduction of external network connectivity that is required, which improves the delivery and response time of the Map for the institutions in that area.

The modularity of the system 100 as is seen for example between the central proprietary sub-system 118, the backup proprietary sub-system 120 and the local proprietary sub-systems 122, enables further sub levels to be provided within the overall system 100 if required. This in turn results in a further reduction of external network connectivity that is required at these sub levels.

Each Map of Medicine Server 124 is configured to specify which external systems it should communicate with e.g. which of the other proprietary sub-systems it should send updates to and accept updates from, which EPRMS systems 106, 108 it can communicate with, which Third Party Knowledge Bases 110 it can access and which healthcare institutions it can provide the Map of Medicine to. The Map of Medicine graphical user interface takes the form of a series of interlinked pages, written in extensible Markup Language (XML), dealing with different health issues which are stored in the Map of Medicine Database 126, each page being identified by a standardised clinical code (e.g. SNOMED-CT codes) for that particular health issue. However, pages from the Map are typically translated into HyperText Markup Language (HTML) by the Map of Medicine Server 124 before being delivered to the browser of the requesting computing terminal 114, 116 (not shown in FIG. 1), since this is the language which most browsers will be configured to use.

The different healthcare institutions, and healthcare practitioners from those institutions, are assigned identifiers against which details of the appropriate version of the Map are stored in the Map of Medicine Database 126. So, for example, a healthcare practitioner using the computing terminal 114 will be provided with the Map of Medicine from the central proprietary sub-system 118, their user-id determining whether they receive the master version of the Map or a local version specified by the healthcare institution with which they are associated. In addition, personalised notes made by healthcare practitioners on nodes within the Map can also be stored in the Map of Medicine database 126 against the user-id. Hence, when a healthcare practitioner requests a particular page from the Map, any personalised notes they have previously made against nodes on that page will be stored in the Map of Medicine Database 126 against their user-id and so can be incorporated into the page that they are provided with. A list of permissions, specifying what actions are allowed by the healthcare practitioner when using Map, will also be stored against their user-id, as will details of paths they have traversed across the Map for training and audit purposes.

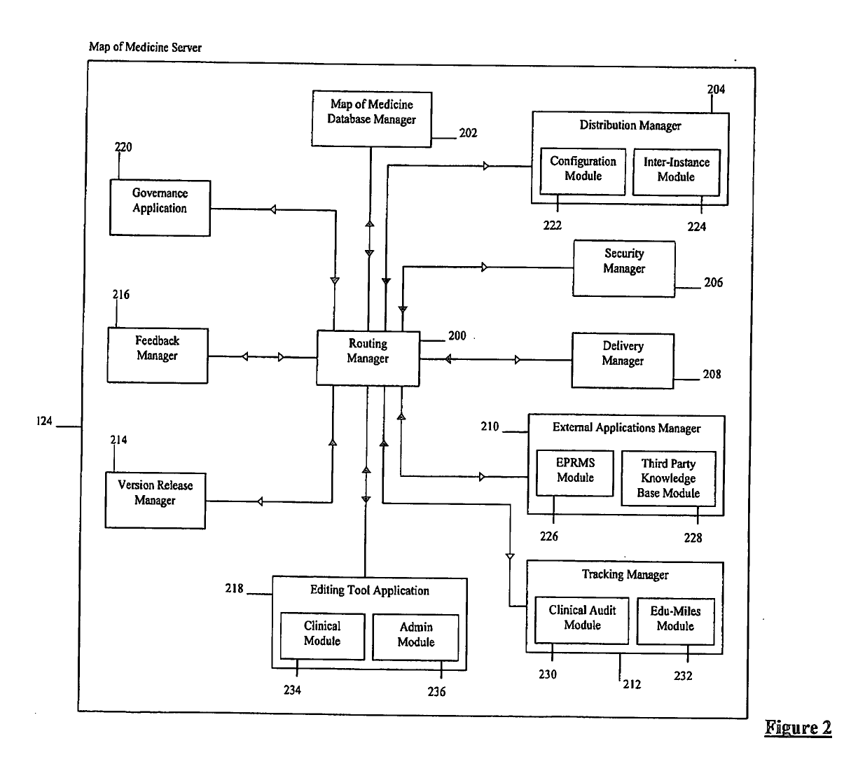

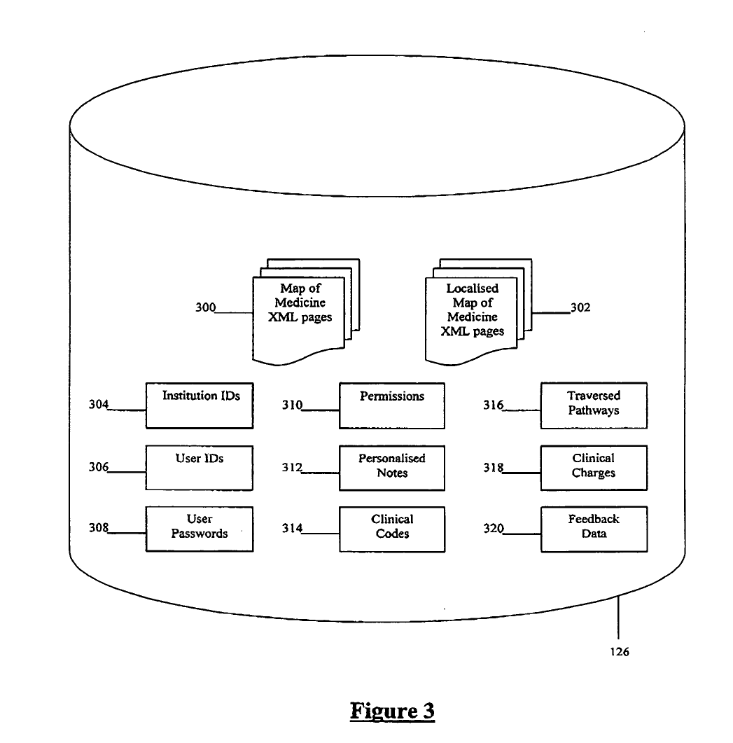

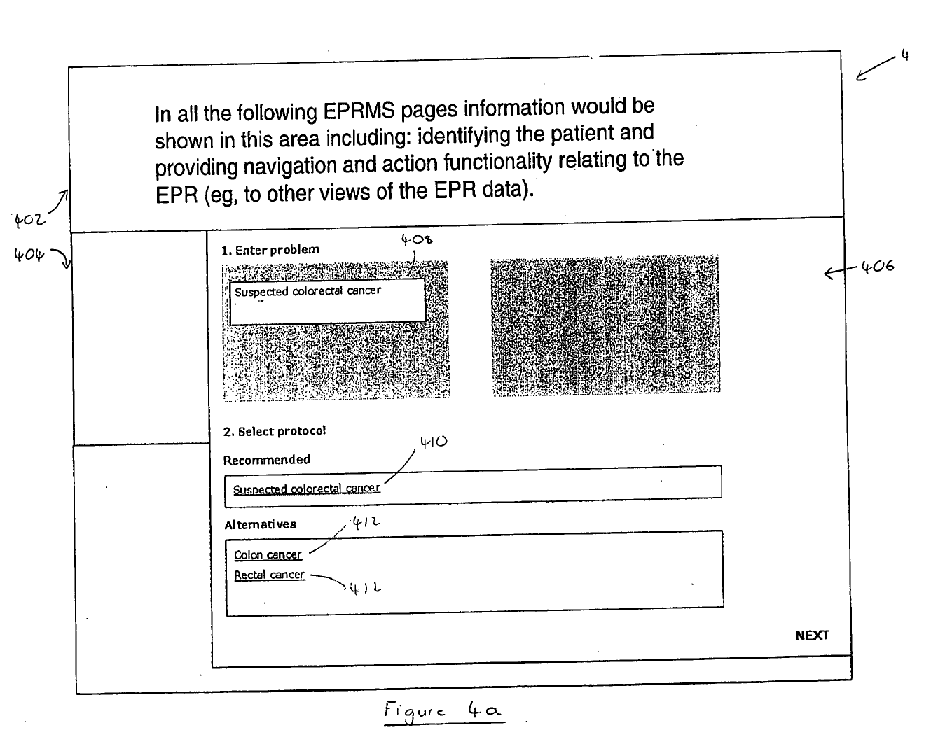

With reference to FIGS. 2 and 3, respectively, the Map of Medicine Server 124 and the Map of Medicine Database 126 of the three proprietary sub-systems 118, 120 and 122 which implement the above functionality will now be described in more detail.

The Map of Medicine Server 124, shown in FIG. 2, is comprised of eleven software processing modules: nine of which are processing managers (a Routing Manager 200, a Map of Medicine Database Manager 202, a Distribution Manager 204, a Security Manager 206, a Delivery Manager 208, an External Communications Manager 210, a Tracking Manager 212, a Version Release Manager 214 and a Feedback Manager 216), which handle internal data processing within the proprietary sub-system 118, 120, 122 and connections with the Communications Network 112 via the NCM 128; and two of which are software applications (an Editing Tool Application 218 and a Governance Application 220), which provide software interfaces through which information stored in the Map of Medicine Database 126 can be accessed.

Communications to and from the Map of Medicine Server 124 are directed to the appropriate software processing module by the NCM 128, as configured by the network communications implementation. The Routing Manager 200 acts as a central hub to which all of the other processing managers and the two software application modules connect, forwarding processing instructions and data to the relevant software processing module. The Map of Medicine Database Manager 202 liases with the Map of Medicine Database 126 under the instruction of the other software processing modules within the Map of Medicine Server 124, handling all queries and updates to the Database 126. A brief description of the general functionality of each of the other processing managers follows below.

Details of any communications received and to be sent by the Map of Medicine Server 124 are forwarded to the Distribution Manager 204, which checks to see if the communication is an authorised one. The Distribution Manager 204 is comprised of a Configuration Module 222 and an Inter-Instance Module 224. The Configuration Module 222 specifies details of external systems that the Map of Medicine Server 124 can communicate with and the functionality that is enabled within that instance of the Map of Medicine Server 124 (not all of the software processing modules may be made available to every instance of the Server 124); it handles the configuration functionality described earlier, namely which other proprietary sub-systems the Server 124 should send updates to and accept updates from, which EPRMS systems 106, 108 it can communicate with, which Third Party Knowledge Bases 110 it can access and which healthcare institutions it can provide the Map of Medicine to. For example, a Local Map of Medicine Server 124 would typically be configured to only send or accept data to or from the central proprietary sub-system 118 and the backup proprietary sub-system 120, whilst the Central Map of Medicine Server 124 would be configured to accept data from all healthcare institutions and local proprietary sub-systems 122 and forward the same to the backup proprietary sub-system 120. Communications with other proprietary sub-systems, such as the management of connections and the scheduling and transfer of data, are handled by the Inter-instance Module 224.

When the Map of Medicine Server 124 receives a request from a healthcare practitioner's computing device 104 to view the Map of Medicine then, after the Distribution Manager 204 has verified that communications from the relevant healthcare institution can be handled, details of the healthcare practitioner's user-id and password will be requested by the Security Manager 206. If the healthcare practitioner is accessing the Map of Medicine directly, then the Security Manager 206 will issue a log-on screen to obtain the relevant details; alternatively, if the healthcare practitioner is accessing the Map of Medicine from within an electronic patient record provided by an EPRMS 106, 108, then the Security Manager 206 may obtain the healthcare practitioner's details directly from the EPRMS 106, 108. The Security Manager 206 forwards the user's details to the Map of Medicine Database Manager 202 so that they can be checked against those stored in the Map of Medicine Database 126. The set of permissions for the healthcare practitioner is returned to the Security Manager 206 and is referred to throughout the user session to determine what the healthcare practitioner can and cannot do in relation to the Map of Medicine. Examples of typical permissions include the right to make changes to the Map of Medicine and the right to accept updates to the Map of Medicine (these will be discussed in more detail later on with reference to the Editing Tool Application 218 and the Version Release Manager 214, respectively).

Once the healthcare practitioner has been verified, the appropriate page of the Map of Medicine corresponding to the request (a home page for the Map of Medicine or a page relating to a particular health issue as specified by a standardised clinical code received in the request) will be retrieved by the Map of Medicine Database Manager 202 and forwarded to the Delivery Manager 208. As mentioned earlier, the Map of Medicine pages as stored in the Map of Medicine Database 126 are written in XML. The Delivery Manager 208 converts the retrieved page from the Map of Medicine into whatever format has been specified by the requesting computing device 104, using standard techniques which are well-known in the art, before transmitting the page to the browser of the device.

The External Applications Manager 210 handles two types of requests which are received by the Map of Medicine Server 124, namely: (1) requests for pages from the Map of Medicine made via an electronic patient record, which are handled by an EPRMS Module 226; and (2) requests, made via the Map of Medicine interface, to connect to external sources of information, which are handled by a Third Party Knowledge Base Module 228. Both of these Modules 226 and 228 use standardised clinical codes (corresponding to diagnoses, symptoms, actions, treatments, operating procedures etc.) to interface with data that is stored outside of the distributed proprietary system 102. The EPRMS Module 226 accesses data from an electronic patient record and uses it to pre-populate corresponding data fields in the page that has been requested from the Map of Medicine for that patient; it uses the clinical codes, embedded against data fields within the Map of Medicine page, to look up data within the electronic patient record, which has been indexed using the same set of standardised clinical codes. Hence, information from a patient's electronic patient record can be contextualised within the Map of Medicine, thereby assisting the healthcare practitioner in their assessment of the patient. Similarly, when a request is made from the Map of Medicine to determine further information about a clinical condition or symptom, the standardised clinical code associated with the condition or symptom is used by the Third Party Knowledge Base Module 228 to identify the relevant information within the Third Party Knowledge Base 110 and take the healthcare practitioner directly to that information. This process does not require any search to be made of the Third Party Knowledge Base 110 which provides faster access to the desired information. Both of these functionalities will be described in further detail in due course, with reference to exemplary screen shots taken from the Map of Medicine interface.

The recording functionality of the Map of Medicine interface is handled by the Tracking Manager 212. Routes traversed by a healthcare practitioner across the Map of Medicine, and actions taken, are recorded by the Tracking Manager 212 and then forwarded to the Map of Medicine Database Manager 202 to be stored against the user-id of the practitioner in the Map of Medicine Database 126. The Tracking Manager 212 additionally comprises a Clinical Audit Module 230, which is used for determining the financial cost of any treatments and actions that are recorded using the Map of Medicine, and an Edu-Miles Module 232 which is used for educational and professional development purposes. The Clinical Audit Module 230 uses the standardised clinical codes that are embedded within the Map, against actions, treatments and operating procedures, to look-up costs associated with those same actions, treatments and operating procedures. In this way, the Map of Medicine allows the cost of the care determined by a pathway through the Map to be quantified. This information can then be made available to an EPRMS 106, 108, such that an invoice for that care can subsequently be generated. The Edu-Miles Module 232 assigns values, or ‘miles’, to routes traversed across the Map of Medicine and information received by the healthcare practitioner, giving an indication of the level of practice to which a healthcare practitioner has been exposed.

The Editing Tool Application 218 allows localised versions of the Map of Medicine to be created, for example through the editing or addition of nodes/pages. Use of these Editing Tool 218 is restricted by permissions. It enables localisation at two different levels, namely the clinical level and the administrative level and these are handled by a Clinical Module 234 and an Admin Module 236, respectively. The Clinical Module 234 facilitates the association of clinical information (such as the definition of a particular condition, the assignment of clinical codes etc.) with particular nodes, whilst the Admin Module 236 allows administrative data fields (such as contact details for a local specialist clinic) to be specified.

The release of any new version of the Map of Medicine is handled by the Version Release Manager 214. The Version Release Manager 214 consults with any localised versions of the Map of Medicine which are stored in the Map of Medicine Database 126 and identifies any areas of conflict; details of the new release and the conflict areas are then forwarded to a Clinical Editor for the healthcare institution to which the localised Map is provided. The Clinical Editor can then either accept the new version, refuse to accept the new version or partially accept the new version, performing a manual integration using the Editing Tool Application 218.

As well as providing work flows to healthcare practitioners, through the Map of Medicine, that are based on best practice guidelines, the present embodiment also facilitates discussion within the healthcare community on the content of those workflows by providing a managed feedback distribution network. Comments which are submitted as feedback via the Map of Medicine interface are distributed by the Feedback Manager 216 to appropriate feedback reviewers, as will be described in more detail later.

Finally, the Governance Application 220 within the Map of Medicine Server 124 can be used to create audit, management and governance reports based on information obtained from the Map of Medicine Database 126. Report elements include assessment of the quantity of localised clinical content implemented in a particular healthcare institution, assessment of the time taken to consider and implement new releases of the Map and assessment of the quantity of feedback being generated from particular healthcare institutions. All of the reporting elements can be implemented using techniques that will be well understood by those skilled in the art of implementing such reporting functions.

The functionality provided by the External Applications Manager 210 and the Tracking Manager 212 will be described in further detail in due course, with respect to a series of exemplary screen shots from the Map of Medicine, screen shots will also be used to further discuss the functionality of the Editing Tool Application 218, whilst the functionality of the Version Release Manager 214 and the Feedback Manager 216 will be discussed with reference to schematic diagrams showing the different hierarchical levels which typically exist within a healthcare system.

However, prior to that, a schematic representation of the type of data stored in the Map of Medicine Database 126 is described with reference to FIG. 3. Further to the above description, it will be apparent that the Map of Medicine Database 126 contains at least the following data elements: the Map of Medicine XML pages 300, although these would only need to be stored by the Central and Backup Map of Medicine Databases; Localised Map of Medicine XML pages 302; Institution IDs 304, as referred to by at least the Distribution Manager 204; User IDs 306, User Passwords 308 and Permissions 310, as referred to by at least the Security Manager 206; Personalised Notes 312, which are added to nodes in the Map by a healthcare practitioner and are provided with those nodes in the Map to that healthcare practitioner thereafter; a set of standardised Clinical Codes 314 corresponding to diagnoses, symptoms, actions, treatments, operating procedures etc. which are used by at least the External Applications Manager 210 when interfacing with eternal data sources and the Clinical Audit Module 230 when monitoring costs; Traversed Pathways 316 detailing routes navigated through the Map of Medicine by healthcare practitioners, as recorded by the Tracking Manager 212; a set of Clinical Charges 318 corresponding to at least some of the Clinical Codes 314, as referred to by at least the Clinical Audit Module 230; and Feedback Data 320, as managed by the Feedback Manager 216. The data elements would be organised within the Map of Medicine Database 126 in a standard manner using conventional data structures, as would be well understood by someone skilled in the art (for example, a database administrator).

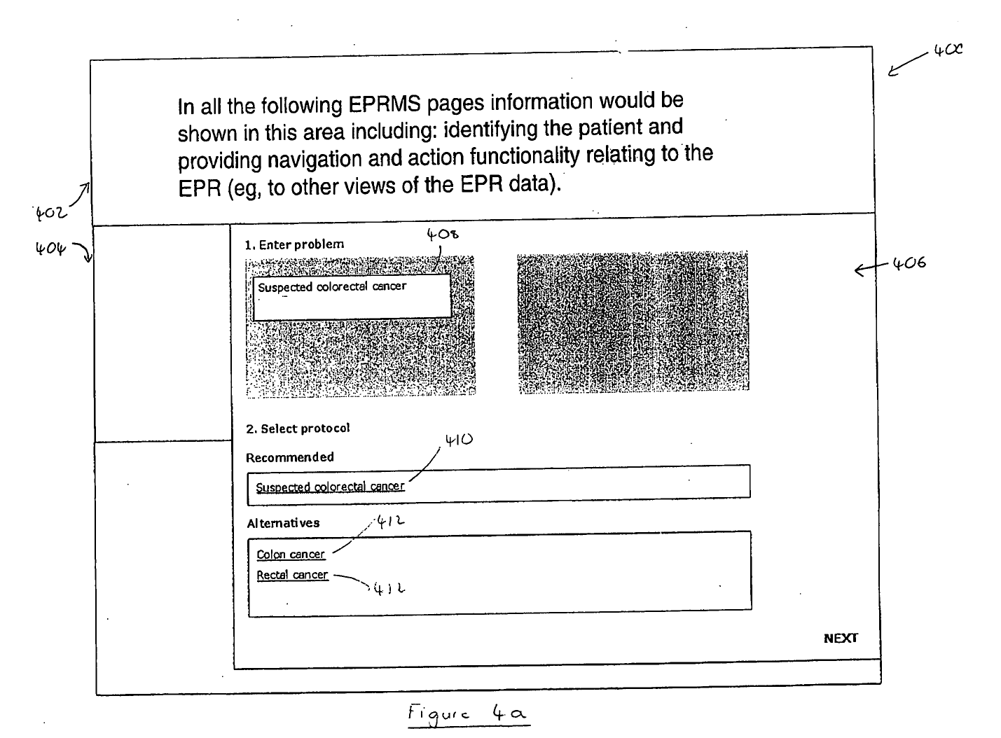

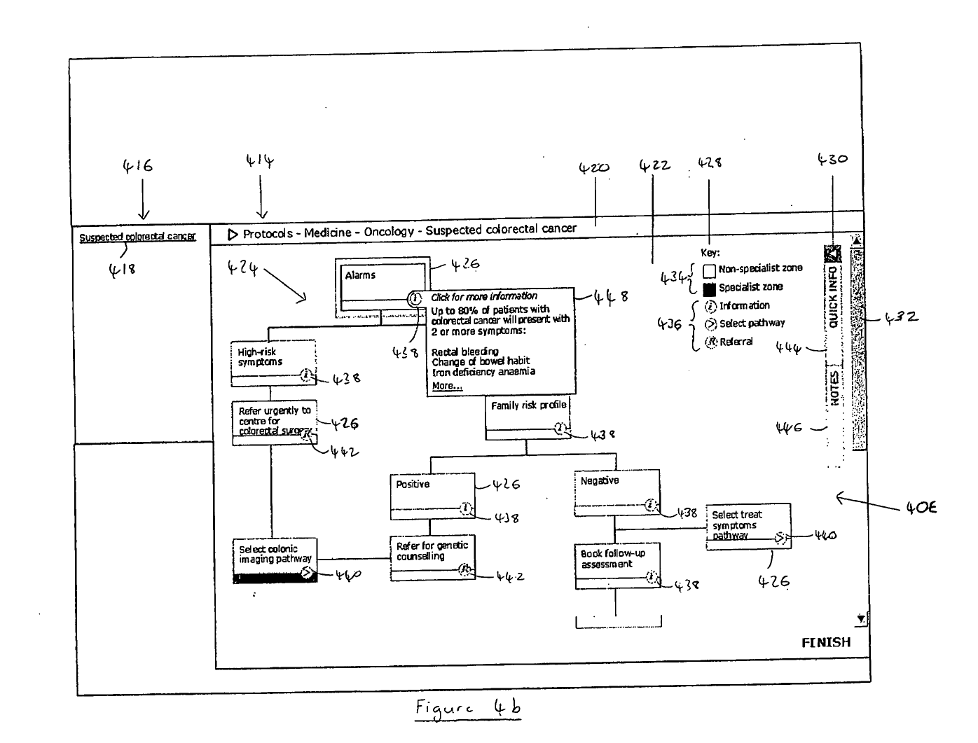

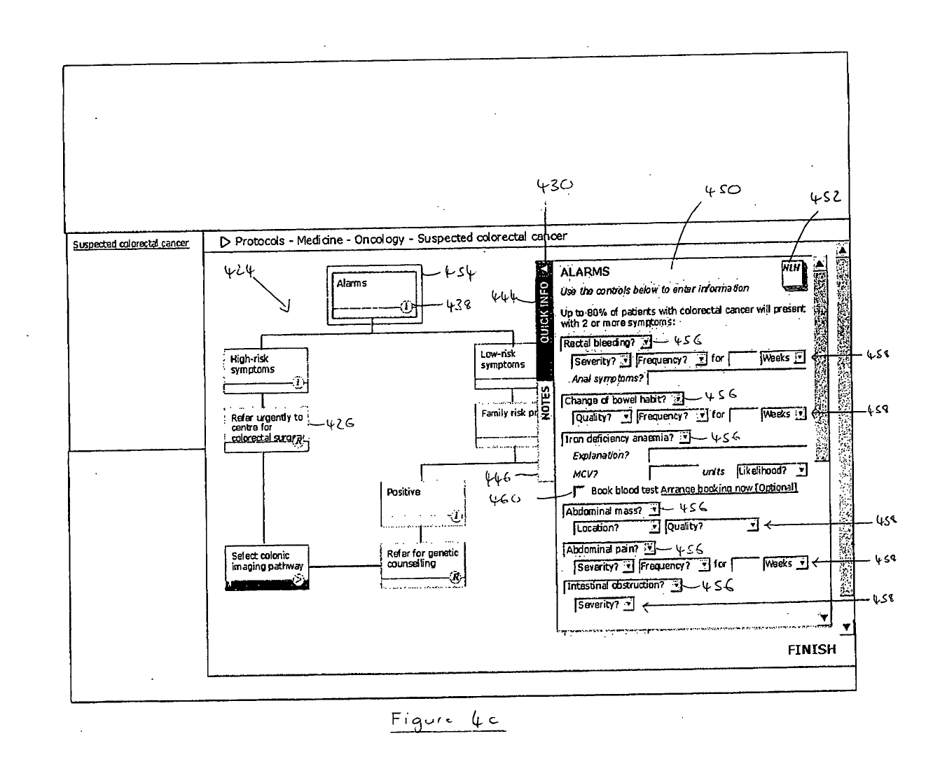

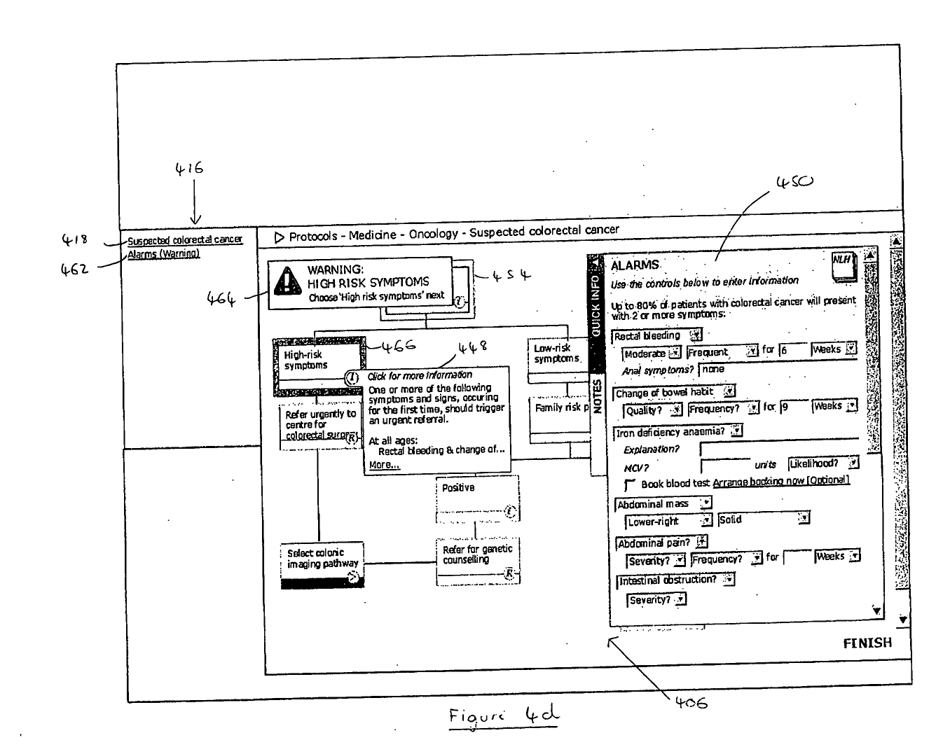

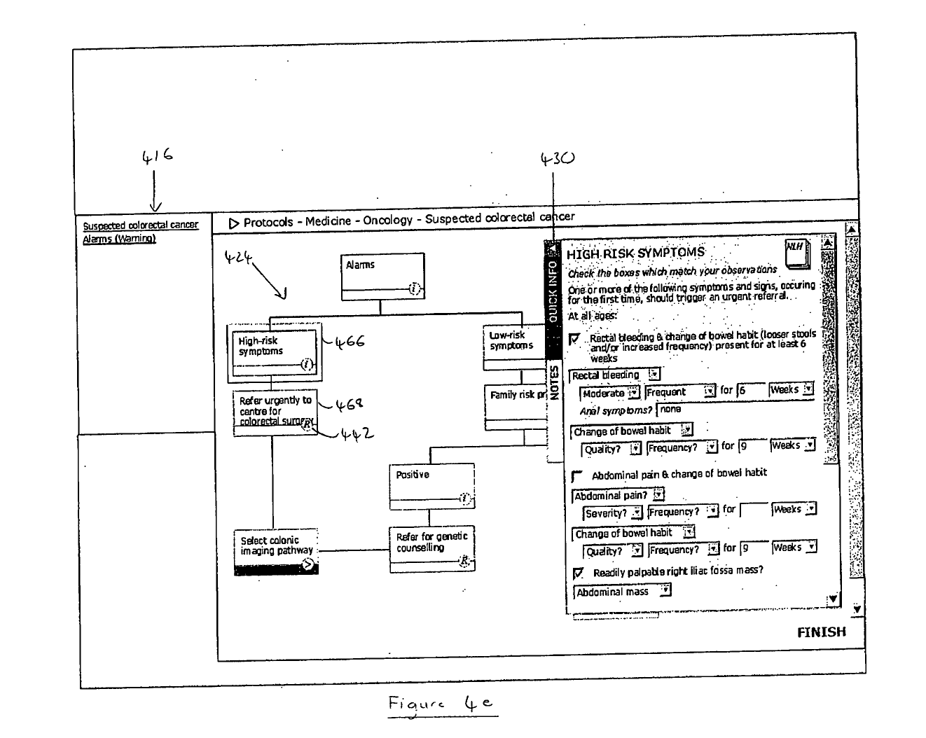

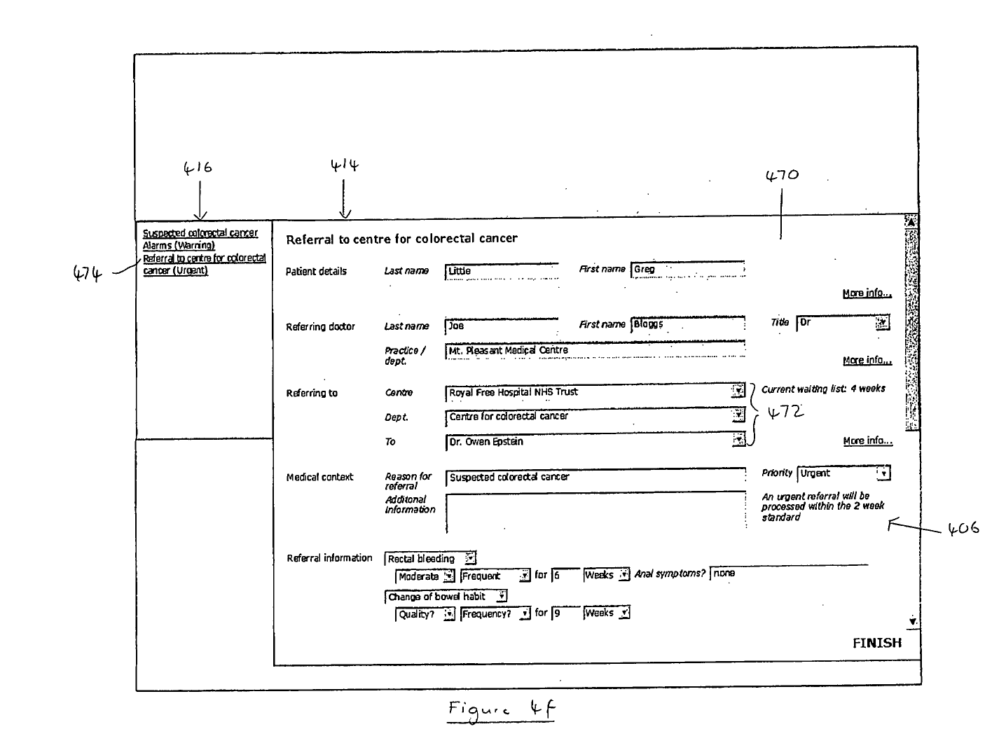

With reference to FIGS. 4a to 4f, a first embodiment of the Map of Medicine GUI, showing the interface functionality when integrated with an EPRMS 106, 108, is now described and the functionality of the EPRMS Module 226 within the Map of Medicine Server 124 is expanded upon. A healthcare practitioner accesses a patient's electronic patient record from an EPRMS 106, 108 using their computing device 104. The GUI provided by the EPRMS includes an option to access the Map of Medicine. On selecting this option the EPRMS GUI 400, as shown in FIG. 4a, is divided into an upper portion 402, which continues to operate under the control of the EPRMS 106, 108, and a lower portion 404 into which the Map of Medicine GUI 406 is inserted. The Map of Medicine GUI 406 fully occupies the area provided by the lower portion 404 and operates under the control of the EPRMS Module 226 within the Map of Medicine Server 124.

The first page of the Map of Medicine which is provided by the EPRMS Module 226 to the computing device 104 contains a problem dialogue box 408, into which the healthcare practitioner can enter details of a healthcare issue (for example, a symptom presented by the patient or a suspected diagnosis). In the present example the healthcare issues under consideration is suspected colorectal cancer. The EPRMS Module 226, on receiving this text from the GUI 406, contacts the Map of Medicine Database 126 to determine the corresponding Clinical Code 314 for that healthcare issue. A link 410 to a recommended page of the Map of Medicine for the healthcare issue, based on the determined Clinical Code 314, is then indicated on the GUI 406, together with possible alternative links 412 (two of which are shown in FIG. 4a) to protocols (workflows) for guidelines on related healthcare issues.

Upon selecting one of the links, the healthcare practitioner is presented with the appropriate page from the Map of Medicine. In the example shown in FIG. 4b, the recommended link 410 has been selected. The GUI 406, when displaying a page from the Map of Medicine, is comprised of a Map navigation portion 414 on the right-hand-side and a pathway recordal portion 416 on the left-hand-side, the pathway recordal portion 416 acting as a margin to the Map navigation portion 414. The pathway recordal portion 416 displays recordable details of the route taken by the healthcare practitioner through the Map of Medicine. This information, including dates and times of when each node was traversed, is forwarded to the Tracking Manager 212 which uploads it to the Map of Medicine Database 126 where it is stored as a Traversed Pathway 316. The upload (actual recordal) can either be done automatically, as and when each node is traversed, or else the actions can be reviewed by the healthcare practitioner when they reach an appropriate point in the workflow, so that changes can be made if so desired (not shown) prior to recordal. Further to the healthcare practitioner selecting the recommended link 410 from FIG. 4a, the pathway recordal portion 416 displays a name 418 for the corresponding page of the Map of Medicine. The Map navigation portion 414, in turn, is comprised of a header portion 420, which identifies the relative location of the present page within the Map of Medicine, and an interactive Map display portion 422.

The Map display portion 422 presents a graphical representation of a pathway or workflow 424 from the Map of Medicine comprising a series of nodes 426, that are linked together in a hierarchical tree structure, the nodes 426 detailing decisions to be made or actions to be taken in respect of the healthcare issue. The displayed workflow representation 424 corresponds to a single page of the Map of Medicine. Also included in the Map display portion 422 are a key 428, a quick information bar 430 and a scroll bar 432. The key 428 defines colour coding 434 that is applied to the nodes 426 (black indicating a specialist zone of the Map, white indicating a non-specialist zone) and a set of interactive icons 436 (namely ‘i’ 438, ‘>’ 440 and ‘R’ 442) that appear on the nodes 426, the functionality of which will be described in due course. The quick information bar 430 is comprised of a quick info tab 444 and a notes tab 446 and allows information to be either quickly entered into the Map by the healthcare practitioner or quickly deduced from Third Party Knowledge Bases 110, as will be described in due course. The scroll bar 432 operates in the standard manner, allowing further nodes 426 from the workflow representation 424, which form part of the page but extend beyond the confines of the Map display portion 422, to be seen.

FIG. 4b also shows what happens when the healthcare practitioner rolls the pointing device of their computing device 104 over an ‘i’ icon 438 that appears on a node 426, namely this action reveals an information text box 448 containing further information associated with that node 426 within the workflow representation 424.

If the healthcare practitioner clicks on the ‘i’ icon 438 with their pointing device, as directed by the information text box 448, then the quick information bar 430 is activated, as shown in FIG. 4c. The quick information bar 430 expands across the screen to reveal an information entry portion 450 and an icon ‘NLH’ 452 linking to Third Party Knowledge Bases 110, collectively referred to as the National Library for Health.

Selecting the quick info tab 444 populates the information entry portion 450 with a questionnaire relating to whatever stage of the workflow representation 424 the node 426 concerns. The information entry portion 450 can additionally be populated with a text box (not shown) allowing local administration information concerning the node 426 to be entered. In the present example, concerning suspected colorectal cancer, the quick information bar 430 has been activated for the root ‘Alarms’ node 454 of the workflow representation 424 which prompts the healthcare practitioner to consider the presentation of certain possible alarm symptoms in the patient. Accordingly, the questionnaire poses a series of questions 456 concerning rectal bleeding, change of bowel habit etc. for the healthcare practitioner to consider when assessing the patient. The healthcare practitioner can record his or her findings in response to the questions 456 by selecting options from one or more drop-down text boxes 458 which appear directly beneath each question 456. The questionnaire also provides an opportunity to schedule a booking at appropriate places within the questionnaire—in the present example, an option for arranging a blood test 460 is shown underneath a question concerning iron deficiency. The EPRMS Module 226 can automatically answer the questions if the relevant information is available from the patient's EPR, although this has not been the case for the example shown in FIG. 4c. Any information entered via the quick info tab 444 is automatically forwarded by the EPRMS Module 226 to the EPRMS 106, 108 for inclusion in the electronic patient record.

In contrast, selecting the notes tab 446 of the quick information bar 430 populates the information entry portion 450 with a text box (not shown) for entering or editing a Personalised Note 312 relating to the issues under consideration in the presently selected node 426. For example, the healthcare practitioner may note details of research they have seen that calls into question the approach dictated by current best practice guidelines. Once a Personalised Note 312 has been added to a node 426, the node is shown on the workflow representation 424 with a note icon (not shown), so that the healthcare practitioner can see which nodes 426 have Personalised Notes 312 against them. The EPRMS Module 226 directs the Map of Medicine Database Manager 202 to store all Personalised Notes 312 against a healthcare practitioner's User ID 306, so that whenever they return to the same workflow representation 424 within the Map of Medicine, their Personalised Notes 312 still appear. Also included in the information entry portion 450 is an option (not shown) for submitting the Personalised Note 312 as Feedback Data 320.

Returning to the present example, FIG. 4d shows the questionnaire associated with the ‘Alarms’ node 454, within the information entry portion 450, after it has been completed by the healthcare practitioner. The pathway recordal portion 416 of the Map of Medicine GUI 406 has been updated to additionally include the name 462 of the ‘Alarms’ node 454 which has been traversed by the healthcare practitioner. The answers provided by the healthcare practitioner in response to the questionnaire have triggered a warning message 464 to be issued on the Map next to the traversed node 454 and mention of the warning is also included in the node name 462 as it appears in the pathway recordal portion 416. The warning message 464 advises the healthcare practitioner which node 426 within the workflow representation 424 to navigate to next, in the present case the ‘High-risk symptoms’ node 466 is the suggested node 466 which is highlighted to bring it to the healthcare practitioner's attention. FIG. 4d shows the healthcare practitioner heeding the warning message 464 and considering the further information associated with the suggested node 466 after having rolled their pointing device over the ‘i’ icon 438 appearing within that node 466.

FIG. 4e shows the Map of Medicine GUI 406 when the healthcare practitioner has activated the quick information bar 430 for the suggested node 466, namely ‘High-risk symptoms’. Again the healthcare practitioner is presented with a questionnaire, but this time some of the answers have been pre-populated based on information provided to the previous node within the workflow representation 424. No further actions are taken with respect to the ‘High-risk symptoms node’ 466 and so its name is not added to the pathway recordal portion 416.

After considering information associated with the ‘High-risk symptoms node’ 466, the healthcare practitioner proceeds to the next stage within the workflow representation 424, namely a node 468 which enables the patient to be referred for surgery. To instigate this, the healthcare practitioner clicks on the ‘R’ icon 442 indicated on the referral node 468 and an appropriate referral form 470 pops up into the Map navigation portion 414 of the Map of Medicine GUI 406, as shown in FIG. 4f. The referral form 470 is pre-populated with information from the electronic patient record by the EPRMS Module 226, whilst the healthcare practitioner can additionally specify to whom the referral should be made by making selections from drop-down text boxes 472 within the form 470. Further to the information being completed, the pathway recordal portion 416 of the Map of Medicine GUI 406 is updated to additionally include the name 474 of the referral node 468.

The remaining icon listed in the key 428, namely the ‘>’ icon 440, links to a different page (workflow representation 424) within the Map of Medicine, concerning a related health issue or continuation of the workflow 424 onto an additional page.

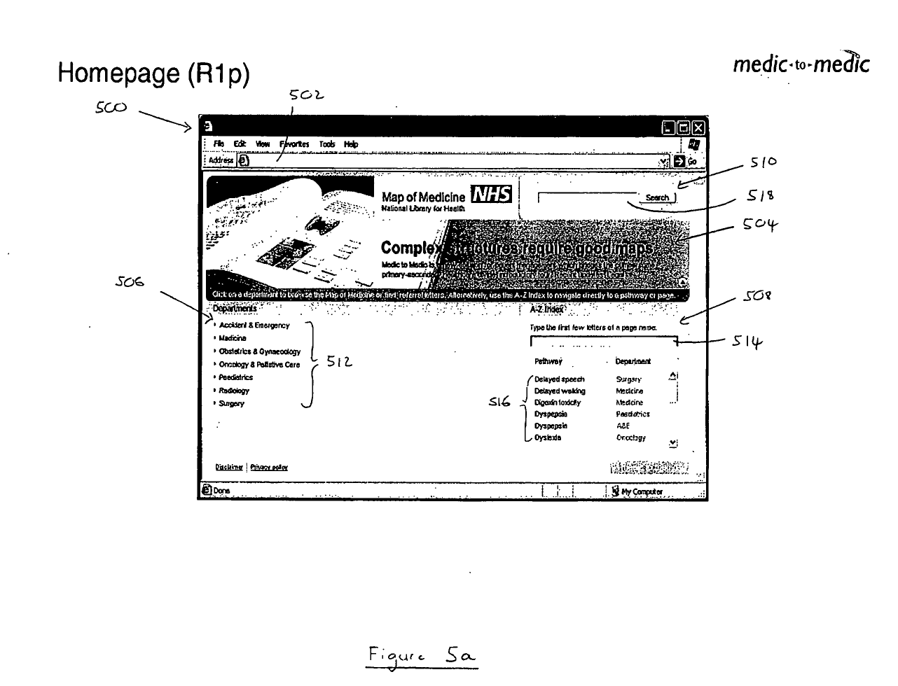

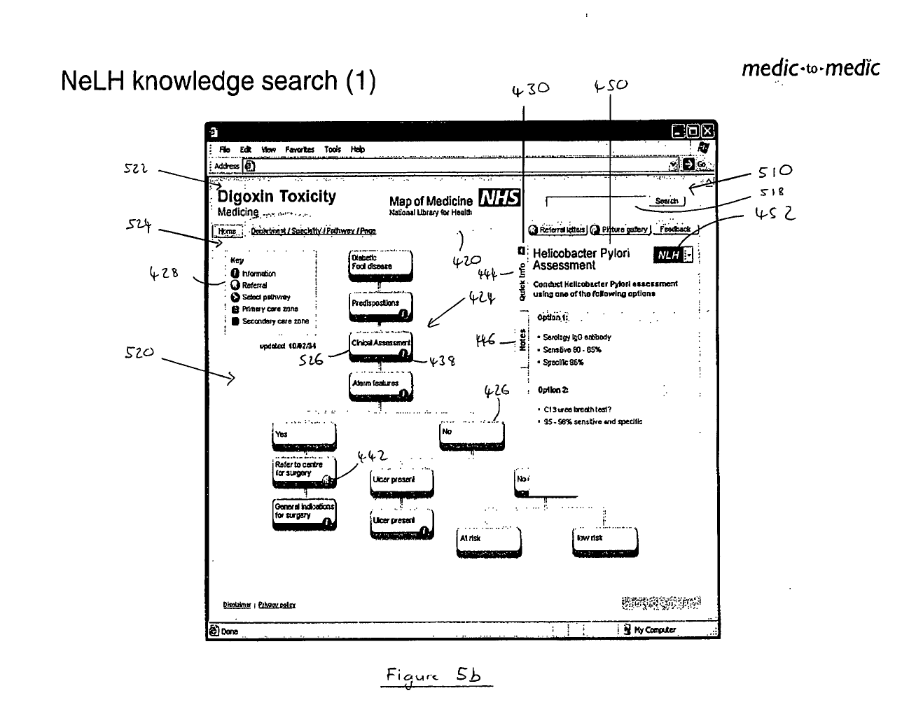

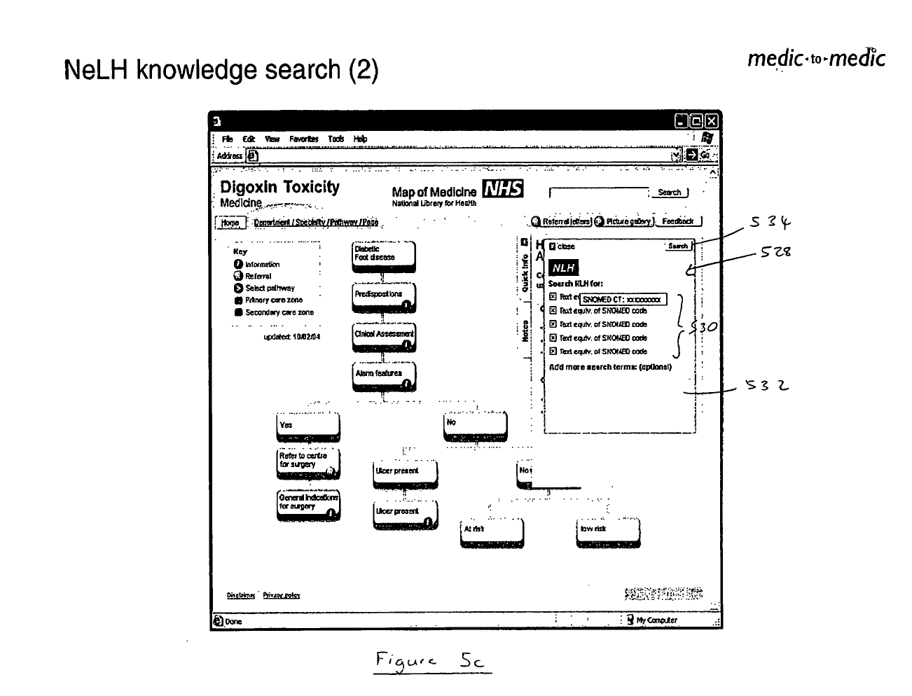

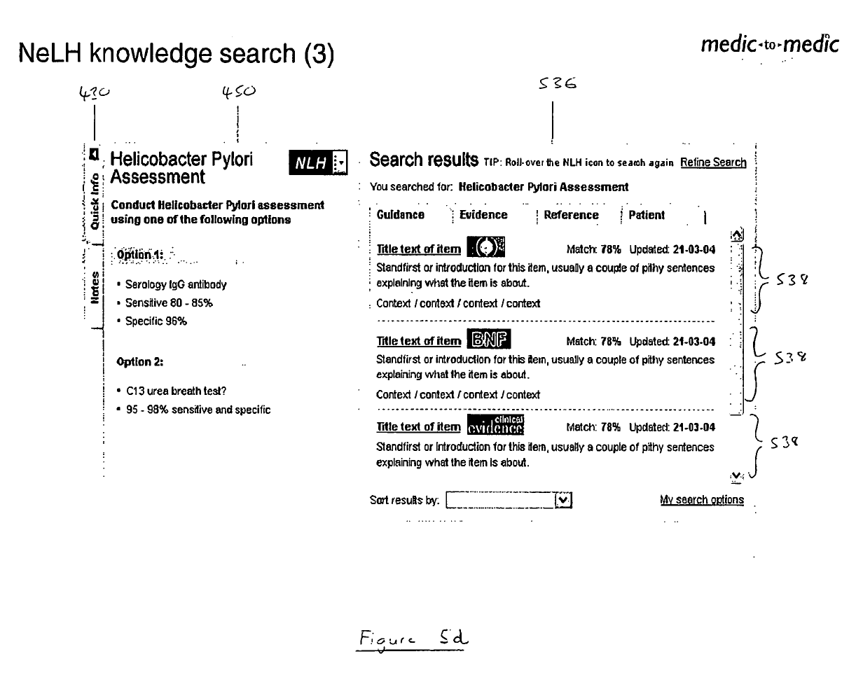

The searching functionality provided within the Map of Medicine will now be described with respect to FIGS. 5a to 5d, according to a second embodiment of the Map of Medicine GUI which is accessed directly rather than through an EPRMS GUI 400. The functionality of the Third Party Knowledge Base Module 228 within the Map of Medicine Server will also be expanded upon.

FIG. 5a shows a browser window 500 displayed on a computing device 104. As mentioned previously, to access the Map of Medicine directly a healthcare practitioner can enter an appropriate URL (not shown) into an address box 502 within their browser window 500. Further to the healthcare practitioner being verified by the Distribution and Security Managers 206 and 208, respectively, within the Map of Medicine Sever 124, a second embodiment of the Map of Medicine GUI 504 would be provided within the browser window 500. The first page provides three different ways in which the workflow representations 424 of the Map of Medicine can be accessed and is comprised of a departments portion 506, an index portion 508 and a general search portion 510. The departments portion 506 contains a set of links 512 to the workflow representations 424 of different healthcare departments. The index portion 508 contains an index box 514 which can be used to search through an alphabetic listing of links 516 to the Map of Medicine workflow representations (pathways) 424. Alternatively the healthcare practitioner can search directly for the workflow representation 424 they require using a search box 518 provided in the search portion 510. In the present example, the healthcare practitioner uses the index portion 508 to select the link 516 for the diogoxin toxixity workflow representation 424 and is presented with the page 520 shown in FIG. 5b.

The page 520 is constructed of a title portion 522, which states the name of the selected workflow representation 424, the search portion 510 described above and a Map navigation portion 524. Unlike in the first embodiment, the Map of Medicine GUI 504 of the second embodiment does not feature a pathway recordal portion 416. However, the Map navigation portion 524 of the second embodiment is very similar to the Map navigation portion 414 of the first embodiment, featuring: a header portion 420; a workflow representation 424; a key 428; and a quick information bar 430, featuring a quick info tab 444 and a notes tab 446, which expands out to reveal an information entry portion 450 and an icon ‘NLH’ 452 linking to Third Party Knowledge Bases 110.

In FIG. 5b, the clinical practitioner has selected the ‘i’ icon 438 on the ‘Clinical Assessment’ node 526 and is being presented with further information on that node 526, namely how to conduct the assessment, rather than a questionnaire as in the previous embodiment. However, the healthcare practitioner can retrieve more detailed information about the assessment via the ‘NLH’ icon 452.

FIG. 5c shows what happens when the healthcare practitioner clicks on the ‘NLH’ icon 452, namely they are presented with a node search dialogue box 528. The Third Party Knowledge Base Module 228 retrieves the text equivalents of the Clinical Codes 314 that are associated with the present node 526 from the Map of Medicine Database 126 and these are presented to the healthcare practitioner in the node search dialogue box 528 as a check-box list 530. Any terms which the healthcare practitioner does not require further information on can be unchecked, whilst any additional terms which the healthcare practitioner wishes to include in the search can be specified in the additional terms text box 532. The healthcare practitioner can commence the search for information on the specified terms by clicking on the ‘Search’ button 534.

Details of the search request are provided to the Third Party Knowledge Base Module 228 which liases with the Map of Medicine Database 126 to obtain Clinical Codes 314 for any additional specified terms, before using the Codes 314 to interface with the Third Party Knowledge Bases 110 (as has been previously described).

In presenting the search results, the Map of Medicine GUI 504 moves the quick information bar 430, together with the information entry portion 450, further across the screen, temporarily obscuring the workflow representation 424, and further expands it by adding a search results portion 536—as shown in FIG. 5d. Summary results 538 from all of the different Third Party Knowledge Bases 110 consulted are listed in the search results portion 536, grouped by category of result, and the full results can be accessed in the usual manner.

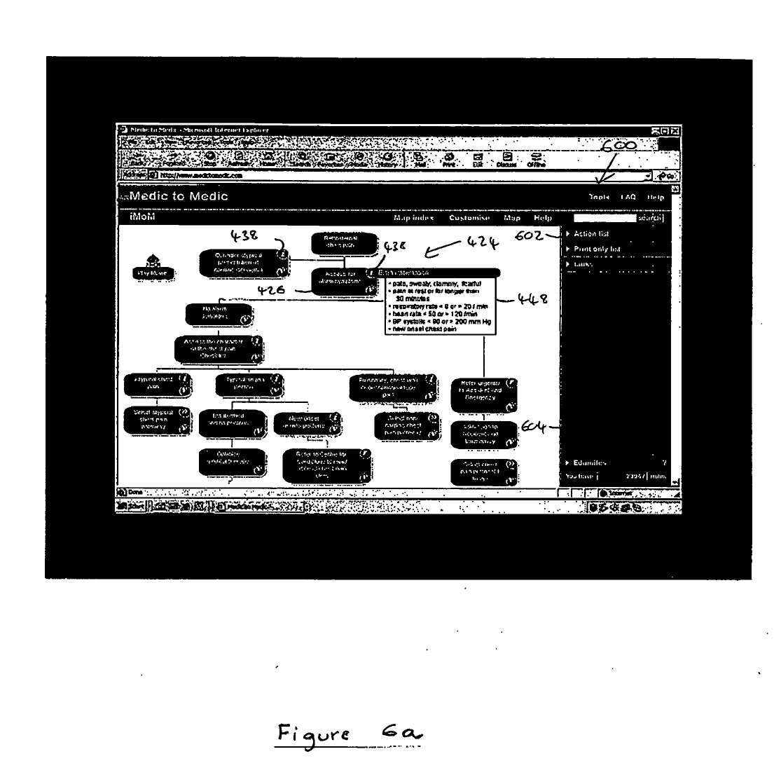

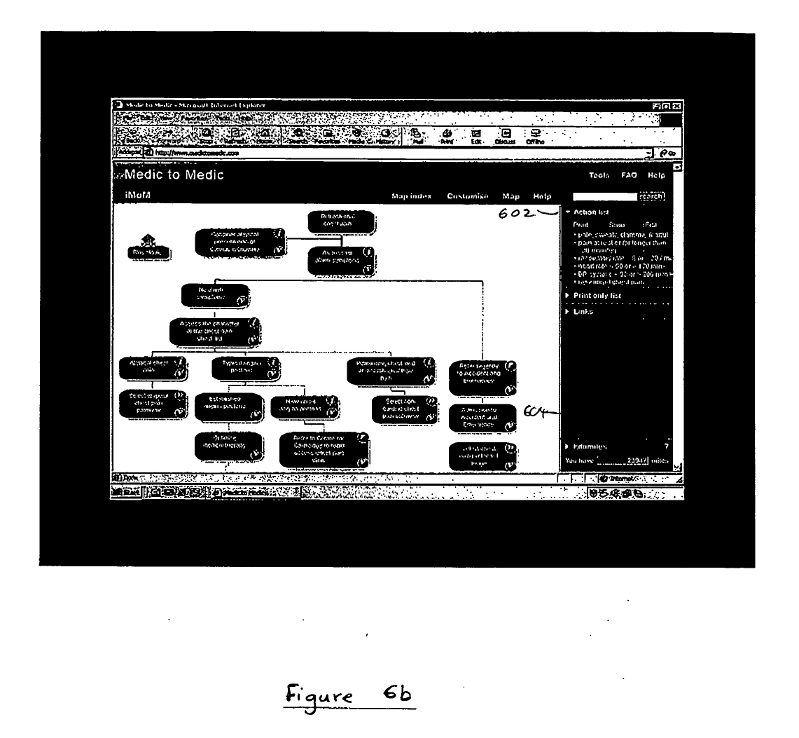

The functionality of the Tracking Manager 212 within the Map of Medicine Server 124 will now be discussed briefly with respect to FIGS. 6a and 6b, according to a third embodiment of the Map of Medicine GUI which is again accessed directly rather than through an EPRMS GUI 400.

A Map of Medicine GUI 600 displaying a selected workflow representation 424 in accordance with a third embodiment is shown in FIG. 6a. In this embodiment information associated with a node 426 can be revealed in a text box 448 in the usual way, namely by a healthcare practitioner rolling their pointing device over the ‘i’ icon 438 on that node 426. However, that same information can be recorded into an Action list 602 by the healthcare practitioner, as shown in FIG. 6b. Options within the Action list 602 enable the healthcare practitioner to print, edit or save the information, the latter option causing the information to be included with the Traversed Pathway data 316 which is recorded by the Tracking Manager 212.

FIGS. 6a and 6b also show output 604 generated by the Edu-Miles Module 232 within the Tracking Manager 212, which measures the healthcare practitioner's exposure to the Map.

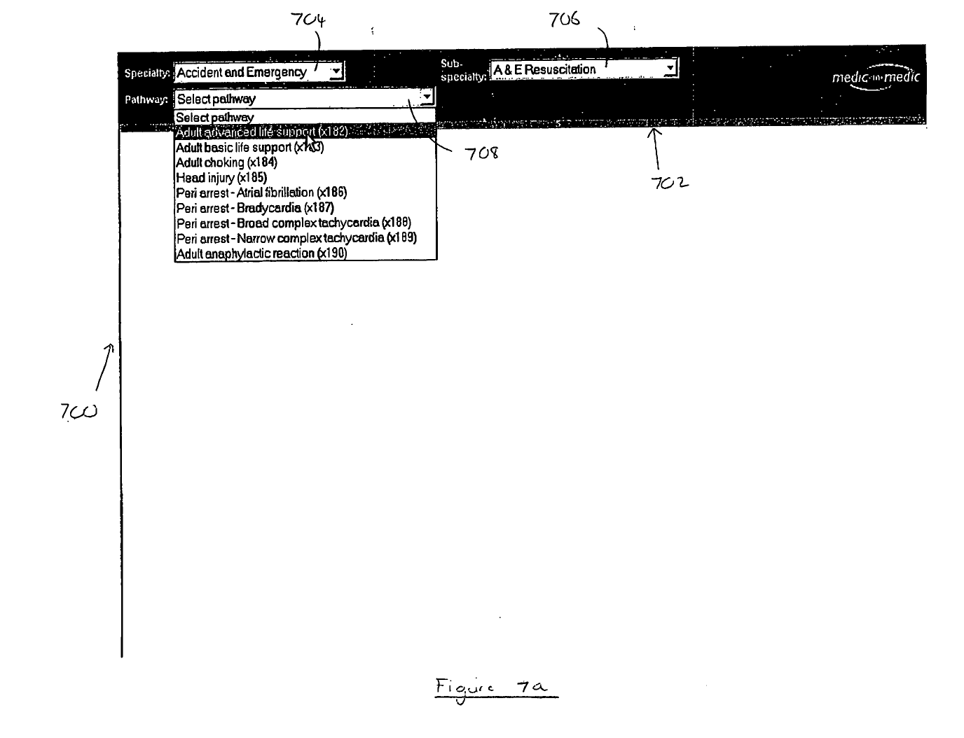

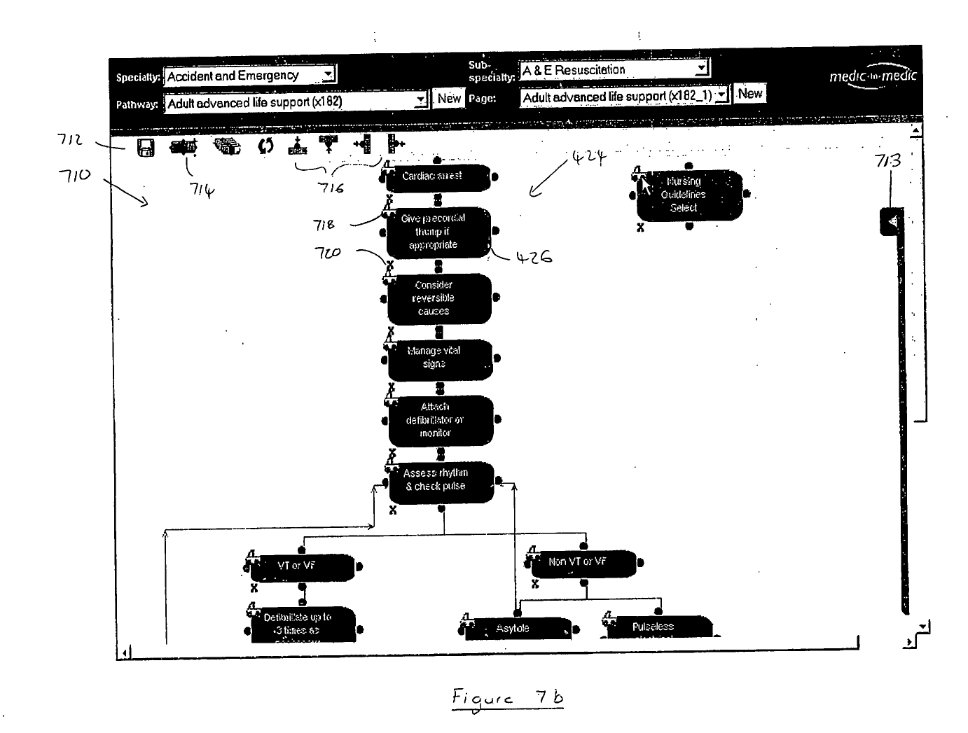

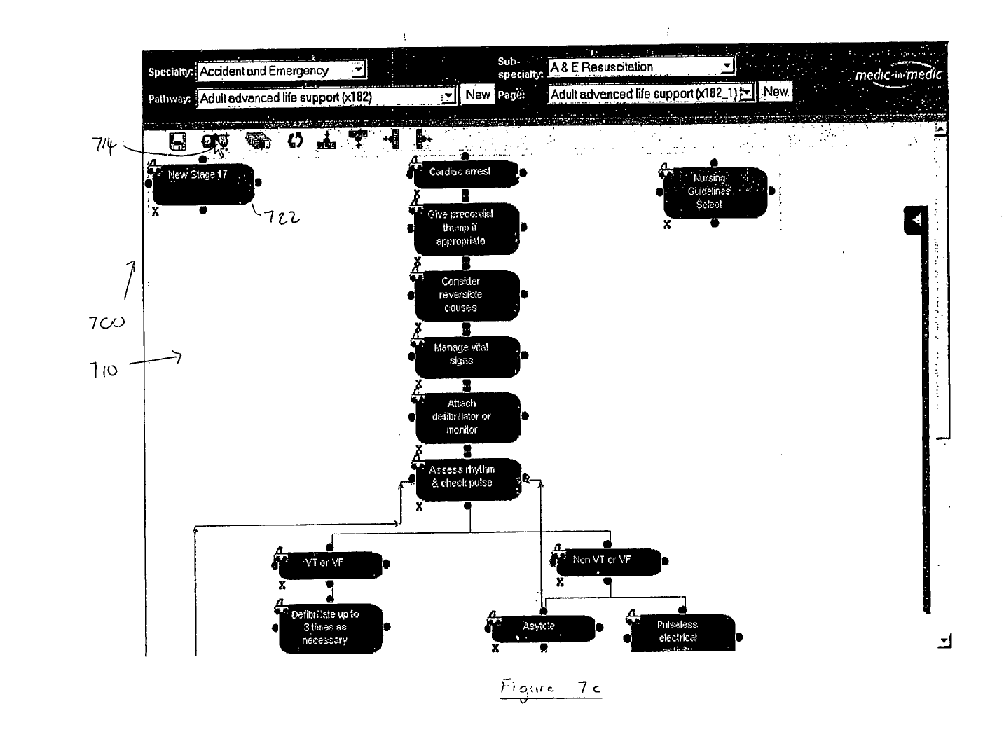

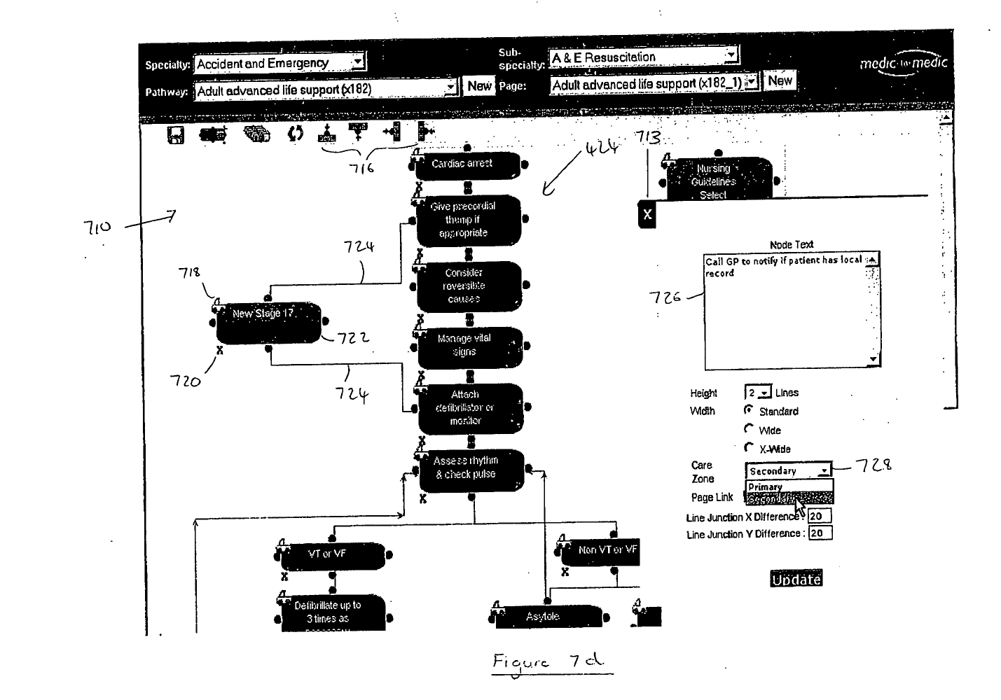

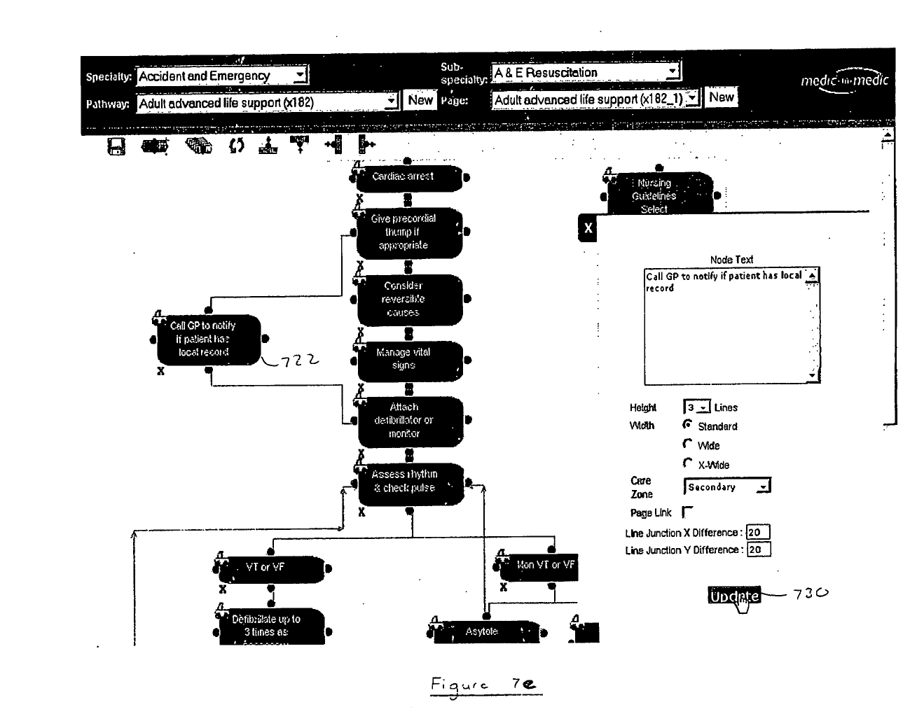

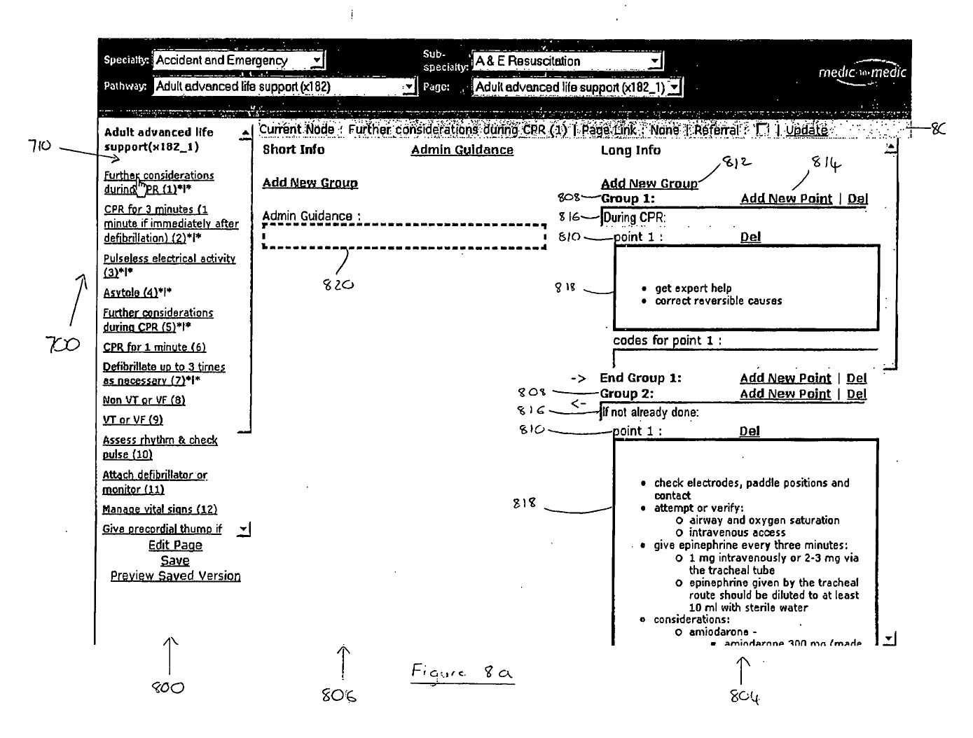

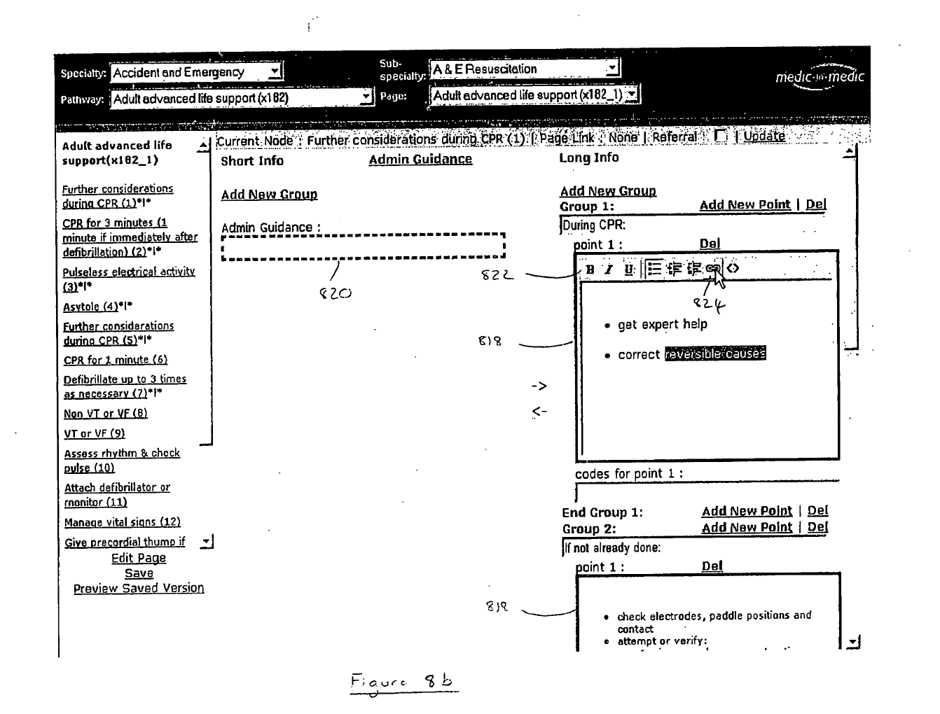

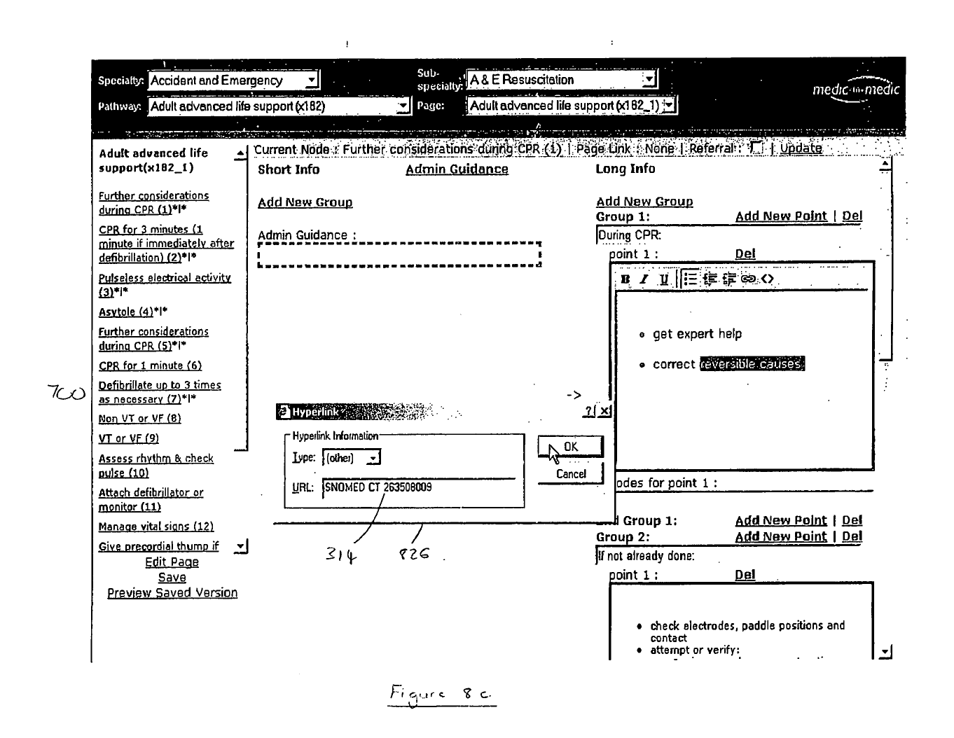

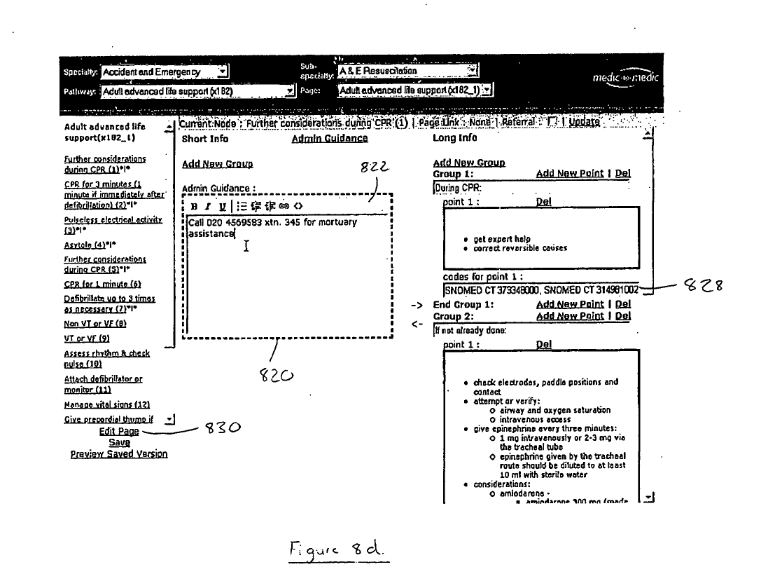

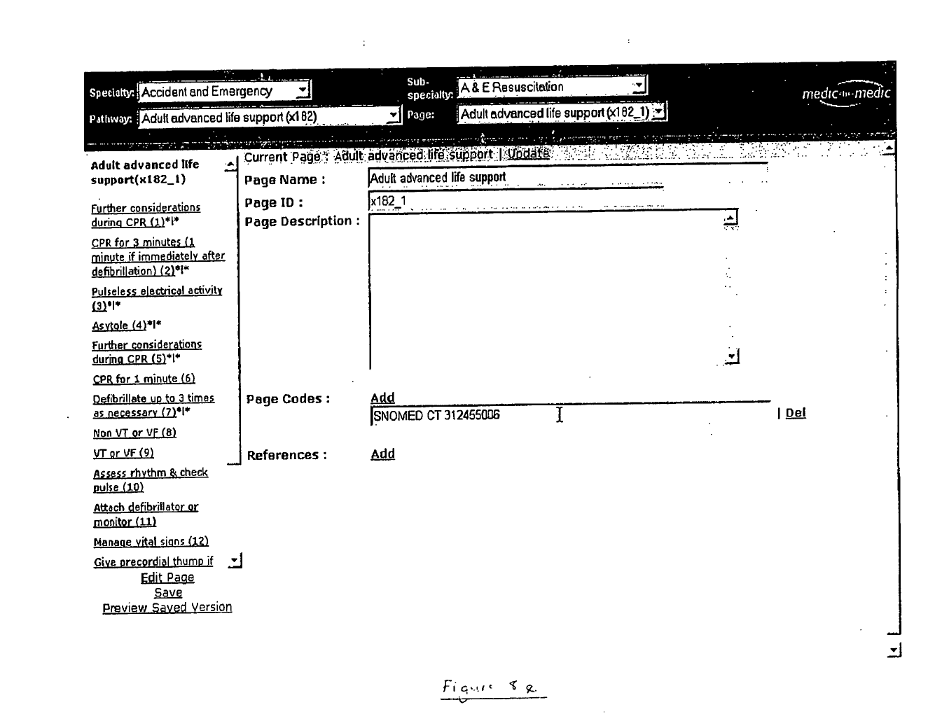

The functionality of the Editing Tool Application 218 will now be described with reference to FIGS. 7a to 7e (which show how the tool can be used to extend an existing workflow representation 424) and FIGS. 8a to 8e (which show how clinical and administration data can be associated with particular nodes 426 in a workflow representation 424).

When the Editing Tool Application 218 is opened by a user, it presents the editing GUI 700 shown in FIG. 7a which is comprised of a navigation bar 702. The user can select a workflow representation 424 (pathway) to edit from the Map of Medicine by making selections from three drop-down boxes within the navigation bar 702, which successively narrow the medical field. The first drop-down box 704 specifies the department, the second 706 specifies the sub-speciality within that department and the third 708 lists the workflow representations 424 that are associated with the sub-speciality of that department.

Further to a selection having been made, the workflow representation 424 is presented to the user in an editing area 710 which lies below the navigation bar 702, as shown in FIG. 7b. A tool bar 712 for editing the structure of the workflow representation 424 is provided at the top of the editing area 710, whilst an expandable bar 713 (via which the appearance of individual nodes 426 can be determined) is provided at the far right-hand-side of the editing area 710. An new node icon 714 for adding new nodes 426 to the workflow representation 424 is provided on the tool bar 712, as are four connector icons 716 for making connections between nodes 426. Two icons are associated with each node 426 appearing within the editing area 710, namely a lorry icon 718 which can be used for moving the node 426 around the editing area 710 and an ‘X’ icon 720 which can be used to remove nodes 426.

FIG. 7c shows the editing GUI 700 after the user has clicked the new node icon 714. A new node 722 appears in the editing area 710. By using its lorry icon 718, the new node 722 can be manoeuvred to an appropriate position within the editing area 710 and then connected to nodes 426 within the workflow representation 424 using connections 724 provided by the connector icons 716, as shown in FIG. 7d. FIG. 7d also shows what happens when the new node 722 is clicked, namely the expandable bar 714 is activated and expands across the screen to reveal a node title text box 726 in which the user can specify the title of the new node 722 and a care zone drop-down box 728, through which the user can specify how the new node 722 should be colour-coded. FIG. 7e shows the selections of FIG. 7d implemented on the new node 722, after the user has committed them using an update button 730.

Information which appears in the information text box 448 of a node 426, which is revealed when a healthcare practitioner rolls their pointing device over the ‘i’ icon 438, is associated with the node 426 by using a content editor, whose functionality will now be described with reference to FIGS. 8a to 8e. The content editor is activated within the editing GUI 700 by a menu option (not shown) and causes the editing area 710 to be divided into a workflow representation listing portion 800, a node header portion 802, a clinical information editing area 804 and an administrative information editing area 806. The clinical information editing area 804 operates under the control of the Clinical Module 234 within the Editing Tool Application 218, whilst the administrative information editing area 806 operates under the control of the Admin Module 236.

The workflow listing portion 800 lists the titles of all of the nodes 426 within the workflow representation 424 that has been selected using the navigation bar 702, beginning with those at the leaf ends of the hierarchical workflow tree structure. In the present example shown in FIG. 8a, the titles from the nodes 426 appearing in the workflow representation 424 of FIG. 7b are listed (the list starts with the titles of nodes 426 which are off-screen in FIG. 7b).

When the user selects one of the node titles from the workflow listing portion 800, the title is written into the node header portion 802. In the example shown in FIG. 8a, the first node title listed has been selected. In addition, any clinical information or administrative information that has already been associated with the selected node 426 is displayed in the clinical and administrative information editing areas 804 and 606, respectively.

Information is entered in the clinical information editing area 804 under group headings 808 as a series of points 810 which are relevant to that group heading 808. Accordingly, the clinical information editing area 804 is provided with a new group operating button 812, new point operating buttons 814, group title text boxes 816 (in which the user can specify the heading text) and point text boxes 818 (in which the user can specify the point that is being made). In contrast, less structure is required for any administrative information that is to be associated with a node 426 and so the administrative information editing area 806 is merely provided with an admin text box 820.

When the user clicks into either one of the point text boxes 818 or the admin text box 820, they are presented with an information editing tool bar 822 as shown in FIG. 8b. One of the icons on this tool bar 822, namely a code association icon 824, allows Clinical Codes 314 to be associated with the information which has been entered in the text boxes 818 or 820. Clicking on this icon 824 causes a code association entry box 826 to appear in the editing GUI 700, as is shown in FIG. 8c.

FIG. 8d shows a series of Clinical Codes 314 appearing in a clinical codes box 828 for the first point 810 under the first group heading 808, the Codes 314 having been entered through the code association entry box 826 which was activated via the point text box 818 for that point 810. FIG. 8d also shows administrative information being entered into the admin text box 820 by the user, the text box 820 having been provided with the information editing tool bar 822 when the user clicked into the same.

Clinical Codes 314 are also be associated with a workflow representation 424 in its entirety as well as its individual nodes 426, as can be seen from FIG. 8e which shows the screen which is arrived at by selecting the ‘Edit Page’ option 830.

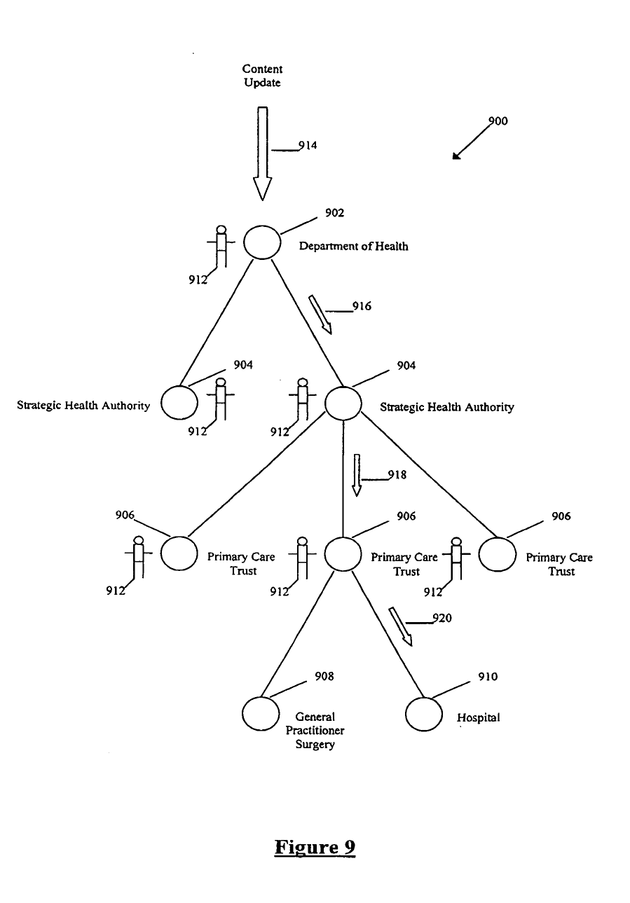

The release of new versions of the Map of Medicine, containing for example new or updated workflow representations 424 which have been edited using the Editing Tool Application 218 described above, will now be described with reference to FIG. 9. In general, healthcare systems are necessarily arranged into different hierarchical levels to assist with their management. Individual hospitals and general practitioner surgeries within a particular local area will be strongly interlinked, with the surgeries referring their patients for treatment at the local hospitals. A body overseeing healthcare provision in the local area may exist to manage the relationship between primary care, at the general practitioner surgery level, and secondary care, provided by the hospitals. Healthcare within a region, covering a plurality of local areas, may benefit from having a single management organisation to implement uniform policy across that region. In the case of a national healthcare system, such as in the UK, all of the regional healthcare management organisations will in turn report to a single governmental department.

The different levels within a hierarchical healthcare structure 900 are represented schematically in FIG. 9. A Department of Health 902, overseeing national health matters, presides at the top of the hierarchical structure 900. A plurality of Strategic Health Authorities 904 (of which only two are shown), overseeing healthcare policy across particular regions, report directly to the Department of Health 902. Each Strategic Health Authority 904 will govern a plurality of Primary Care Trusts 906, which oversee healthcare relationships within local areas of the region. In FIG. 9, only three Primary Care Trusts 906 for one of the Strategic Health Authorities 904 are shown. At the lowest level of the hierarchical healthcare structure 900, FIG. 9 shows only a single General Practitioner Surgery 908 and a single Hospital 910 which fall under the ‘umbrella’ of one of the Primary Care Trusts 906.

As has been mentioned previously, when an updated version of the Map of Medicine is to be released to a healthcare institution, the Version Release Manager 214 within the Map of Medicine Server 124 accessed by that healthcare institution identifies any areas of conflict with the localised version of the Map for that institution and brings these areas to the attention of a Clinical Editor for the institution. In FIG. 9, a Clinical Editor 912 is stationed at every level within the hierarchical healthcare structure 900 bar the lowest one containing the General Practitioner Surgery 908 and the Hospital 910 which, in the present example, are not provided with the necessary Permissions 310 to implement their own changes to the Map of Medicine.

In the present example, a new version of the master copy of the Map of Medicine is released by the proprietor of the distributed system 102 to the central proprietary sub-system 118. The Version Release Manager 214 within the Central Map of Medicine Server 124 identifies the changes between the present master copy of the Map and the new version and notifies the Clinical Editor 912 stationed at the Department of Health 902 of the same. The Clinical Editor 912 can then either accept the new version or refuse to accept the new version. It is also possible for the Clinical Editor to partially accept the new version by performing a manual integration of some parts using the Editing Tool Application 218. For the purposes of the present example, we will assume that the changes are accepted in full, so that employees within the Department of Health 902 are subsequently provided with pages from the updated master copy of the Map of Medicine. This action causes the Version Release Manager 214 to process the release for the next level within the hierarchical structure 900, namely the Strategic Health Authorities 904. One of the Strategic Health Authorities 904 has its own localised version 302 of the Map of Medicine which is stored in the Central Map of Medicine Database 126. Accordingly, the Version Release Manager 214 identifies the differences between the new master copy of the Map (accepted by the Department of Health 902) and the localised version 302 used by the Strategic Health Authority 904, automatically incorporates the local preferences where they do not present a conflict but notifies the Clinical Editor 912 stationed at that Strategic Health Authority 904, via the flow step 916, of any areas of conflict with previously implemented local changes. After consulting with colleagues, the Clinical Editor 912 uses the Editing Tool Application 218 to manually edit the new version of the localised Map into an acceptable form and it is this version which will be subsequently accessed by employees within the Strategic Healthcare Authority 904.

The next level of the hierarchical healthcare structure 900 is populated by the Primary Care Trusts 906. The Version Release Manager 214 notes that one of these accesses its Map of Medicine from its own local proprietary sub-system 122. Accordingly, the Version Release Manager 214 within the central proprietary sub-system 118 forwards a new copy of the Map of Medicine, which the Strategic Health Authority 904 deemed acceptable, to the local proprietary sub-system 122, as is indicated by flow step 918 in FIG. 9.

The Version Release Manager 214 within the Local Map of Medicine Server 124 of the Primary Care Trust 906 implements variations from the local Map which present no conflict into the new Map and then goes on to inform the local Clinical Editor 912 of those areas where there are conflicts. These areas are resolved by the local Clinical Editor and a new version of the localised Map is implemented, such that when the Hospital 910 in the next level down in the hierarchy 900 requests a page from the Map, it is provided with a page from the new localised version, as indicated by the flow step 920.

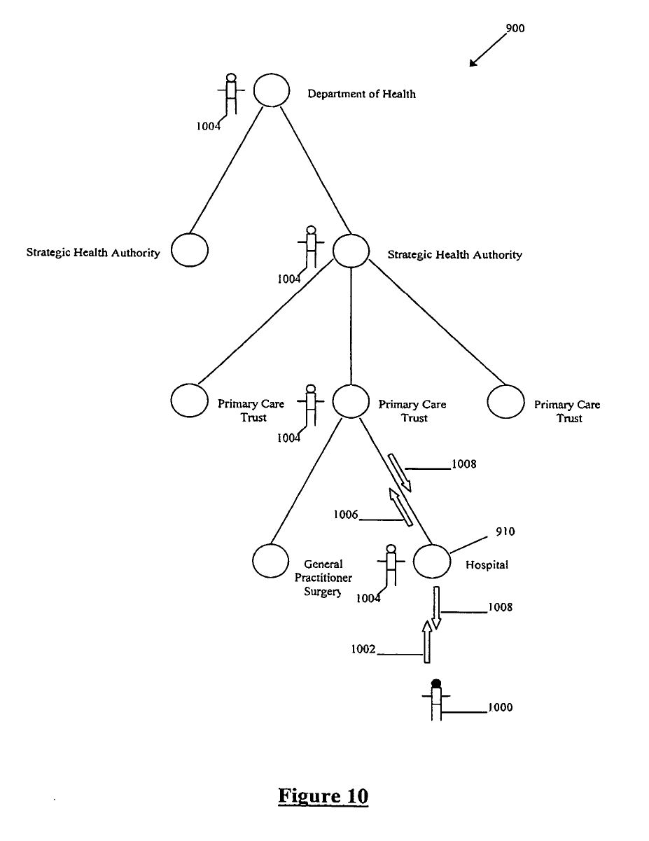

The hierarchical healthcare structure 900 described above in relation to version release management, will now also be used in FIG. 10 to describe the functionality of the Feedback Manager 216 within the Map of Medicine Server 124. Whilst healthcare practitioners can readily agree on best practice guidelines in the face of sufficient research and evidence, most of medicine is based on consensus and expert opinion. As well as providing healthcare practitioners with graphical representations of workflow representations 424, which accord with best practice guidelines, the present embodiment also provides a managed network through which feedback on the content of the workflow representations 424 can be distributed.

As has already been described, feedback relating to a particular node 426 within a workflow 424 can be submitted from the Map of Medicine GUI 406 by a healthcare practitioner 1000 using the notes tab 446 on the quick information bar 430. The healthcare practitioner 1000 can draft a Personalised Note 312 and then select an option which submits the note to the Map of Medicine Server 124. The note is then forwarded to the Feedback Manager 216 within the Map of Medicine Server 124 which provides the Map pages to that healthcare practitioner 1000, as indicated in flow step 1002, and stored as Feedback Data 320 in the Map of Medicine Database 126.

Feedback Reviewers 1004 for different medical departments and specialities within those departments are assigned at each level of the hierarchical structure 900 and the Feedback Manager 216 is provided with the User ID 306 of each Reviewer 1004. Accordingly, upon receiving the Feedback Data 320 from the healthcare practitioner 1000, the Feedback Manager 216 notes from which node 426 the information originated, looks up the Feedback Reviewer 1004 within the same institution as the healthcare practitioner 1000 for that node 426 (in the present example, the healthcare practitioner is located in the hospital 910) and forwards the Feedback Data 320 to that Feedback Reviewer 320, who is alerted by e-mail. The e-mail directs the Feedback Reviewer 1004 to a feedback summary page (not shown) within the Map of Medicine where they can assess the issue raised in the feedback.

If the Feedback Reviewer 1004 cannot answer the query, then they have a responsibility to direct it to the Feedback Reviewer 1004 at the next level up within the hierarchical structure 900 for that medical speciality. After this option from the summary page has been selected, the Feedback Manager 216 identifies the relevant Feedback Reviewer 1004 and notifies them by e-mail (as indicated by the flow step 1006); it also notifies other people within the feedback chain that the issue has been forwarded and then records this action in the feedback summary page. At any time, any person in the feedback chain relating to a particular query can access the feedback summary page and see details of the progress being made. Hence, in the present example, the responsibility for answering the query now rests with the Feedback Reviewer 1004 at the Primary Care Trust 906.

Similarly, if a Feedback Reviewer 1004 replies to the feedback via the feedback summary page, then the Feedback manager 216 notifies everyone in the feedback chain who can then view the reply on the feedback summary page. In FIG. 10, a reply is indicated by the flow steps 1008.

Having described particular preferred embodiments of the present invention, it is to be appreciated that the embodiments in question are exemplary only, and that variations and modifications, such as those that will occur to those possessed of the appropriate knowledge and skills, may be made without departure from the spirit and scope of the invention as set forth in the appended claims.

The communications system 100, shown in FIG. 1, could be structured in a variety of ways. For example, the second computing terminal 116 could be connected to the Local EPRMS 108 directly through a local network, rather than via the Communications Network 112. The local Map of Medicine Server 124 could be configured to access data from the Central EPRMS 106 as well as the Local EPRMS 108. It would also be possible, using so-called ‘grid’ methodology, for different instances of the local proprietary sub-systems 122 to access the Maps of Medicine stored by other local instances. So, for example, a healthcare practitioner who was temporarily seconded to a hospital in a different region would still be able to access their ‘home’ version of the Map of Medicine. Furthermore, the Central EPRMS 106 could be made redundant if data from Local EPRMSs 108 was accessible to all other local proprietary sub-systems 122 via the ‘grid’ methoology. In addition, a Local Map of Medicine Database 126 for a region could store the local Maps of Medicine for a plurality of hospitals in that region. Indeed, it is possible for localised versions of the Map of Medicine to be stored for individual healthcare practitioners, but this would not promote harmonised patient care and so is not favoured by presently preferred embodiments. Of course, the communications system 100 could be simplified by not having any local proprietary sub-systems 122. Furthermore, it will be apparent that the Map of Medicine pages can be provided by the Delivery Manager 208 to a range of computing devices 104, including those which operate via mobile telecommunications protocols such as a personal digital assistant.

It is also feasible that the Map of Medicine could be made accessible to a healthcare practitioner in a number of different ways. For example, if the healthcare practitioner is familiar with the Clinical Code 314 of a symptom/diagnosis, then they could enter this directly into the problem dialogue box 408 shown in FIG. 4a and be provided with the relevant workflow 424 from the Map. Alternatively, rather than having to specify a particular healthcare issue, the healthcare practitioner could be taken to a page within the Map of Medicine for a patient based on the most recently specified Clinical Code 314 within the patient's electronic patient record. Another possibility would be for the healthcare practitioner to be presented with a summary page listing workflows 424 previously traversed for the patient; the healthcare practitioner could select the relevant workflow 424, be informed of the nodes traversed therein to date, and then continue with the patient journey. It is also conceivable that an EPRMS 106, 108 could be accessed through the Map of Medicine rather than vice-versa.

The route taken through the Map could be distinguished in some way, as and when nodes 426 are selected. For example, the nodes 426 themselves could be highlighted in some way, or else the connections between them could. Additional means could also be used to indicate the path taken—for example a series of arrows could be overlaid on top of the selected nodes 426. In some cases, it may also be possible for the clinical practitioner to skip certain nodes 426 in a workflow 424, this functionality being incorporated into the definition of the node.

It is also envisaged that information which is incorporated into the Map of Medicine from a patient's electronic patient record would have some sort of time-limit processing applied to it by the EPRMS Module 226. For example, details of rectal bleeding recorded five years ago, may not be relevant to an assessment of colorectal cancer in the present.

For training purposes, the Map of Medicine could be implemented against a database of dummy patient data to create a simulated EPRMS environment. With regard to both training and monitoring professional development of healthcare practitioners, the Edu-Miles Module 232 could be configured to only award ‘miles’ in respect of any new area of the map which is traversed.

In particular, it will be appreciated that whilst specific embodiments of the Map of Medicine GUI have been described hereinbefore, features from the different embodiments can be combined in a variety of ways to create novel interfaces which are also within the scope of the invention.

Variations within the Editing Tool Application 218 are also possible. For example, it is envisaged that Clinical Codes 314 will be able to be assigned automatically to nodes 426, thereby removing the need for the code association process shown in FIG. 8c. Aspects of the version release management process, for example the manual merging process outlined in the description, could also be the subject of automation in due course.

Finally, it will be appreciated that the invention is not restricted to implementation in a healthcare environment; rather it can be applied to any environment where data entry from an interlinked series of workflows is required.

1. A graphical user interface (GUI) for interacting with a user during a workflow process, the GUI comprising:
a page including a plurality of interlinked nodes which graphically represent the structure of a plurality of interlinked steps of a stored workflow process;
data entry means for entering data relating to a particular selected node; wherein the node has a unique relationship with a step in the workflow process;
pathway means for determining a particular path through the workflow process using the entered data; and
means for graphically representing the resultant path through the workflow process in the page.

2. A GUI according to claim 1, wherein the plurality of interlinked nodes represent a complete workflow process on a single page.

3. A GUI according to claim 1 or 2, wherein each node represents an action, decision or result within the workflow process.

4. A GUI according to claim 1, wherein the data entry means comprises presentation means for presenting data relevant to a location of the selected node within the plurality of interlinked nodes and selection means for enabling user selection of at least some of that data.

5. A GUI according to claim 4, wherein the presentation means comprises a plurality of drop-down lists of location-specific information.

6. A GUI according to claim 1, wherein the data entry means is arranged to use the entered data at a first node to determine further information required at a second node, linked to the first node.

7. A GUI according to claim 1, further comprising updating means for updating any information related to the step in the workflow process with entered data.

8. A GUI according to claim 1, further comprising means for converting the entered data into a classification code representing that data.

9. A GUI according to claim 8, wherein the classification code comprises a standard classification code describing a complete range of possible data inputs relevant to the subject of the workflow process.

10. A GUI according to claim 8, wherein the subject of the workflow process is clinical medical information and the classification code represents one of the group comprising a diagnosis, a symptom, an action, a treatment and an operative procedure.

11. A GUI according to claim 1, further comprising analysing means for analysing the entered data and generating a list of actions associated therewith and listing means for listing the list of associated actions to the user adjacent the plurality of displayed interlinked nodes.

12. A GUI according to claim 1, further comprising an information means provided at a node for presenting information associated with a node upon user selection.

13. A GUI according to claim 12, wherein the information means comprises a graphical icon and user selection comprises interaction between a user navigational tool and the icon.

14. A GUI according to claim 12, wherein the information means is arranged to provide a plurality of different levels of detail of information, in accordance with a user selection.

15. A GUI according to claim 1, further comprising action list means for generating a list of actions and presenting the same to the user adjacent the plurality of displayed interlinked nodes, the action list means being arranged to determine the list from analysis of the user navigation through the plurality of interlinked nodes.

16. A GUI according to claim 15, wherein the action list means is arranged, at the end of traversal of a plurality of interlinked nodes comprising the page, to present the list to the user with options for user confirmation of each action, and to determine the list of actions to be implemented from the user confirmation.

17. A GUI according to claim 1, further comprising a note recordal means for recording user-generated textual note relating to a particular node, the note recordal means being arranged to link the note with the particular node such that the stored note is retrievable when the user has navigated to that particular node.

18. A GUI according to claim 17, wherein the note recordal means is arranged to record a user-determined variation of the workflow process at a particular node.

19. A GUI according to claim 17, further comprising feedback generation means for converting a user-determined note into a transmittable message and for transmitting the message to another user having access to a version of the GUI.

20. A GUI according to claim 1, further comprising a new page linking means for linking a node at the end of a branch of the plurality of interlinked nodes within one page to a node within another different page.

21. A GUI according to claim 20, wherein the new page linking means comprises a graphical icon and user selection comprises interaction between a user navigational tool and the icon.

22. A GUI according to claim 1, wherein the GUI has access to an Electronic Patient Record Management System and the GUI further comprises an EPRMS management means for obtaining and presenting details of a selected electronic patent record in a portion of the page.

23. A GUI according to claim 22, wherein the EPRMS management means further comprises population means for populating one or more nodes with at least some of the details of a selected electronic patent record, thereby reducing any required data entry at that node.

24. A GUI according to claim 22, wherein the EPRMS management means is arranged to use the details of the selected electronic patient record to determine what information is required at a node from the user.

25. A GUI according to claim 1, further comprising referral means for generating a referral message, the referral means being provided at a node and using information associated with the node to populate at least some of the referral message on user-selection.

26. A GUI according to claim 25, wherein the referral means comprises a graphical icon and user selection comprises interaction between a user navigational tool and the icon.

27. A GUI according to claim 25, wherein the referral means is arranged to use information obtained from an electronic patient record to populate automatically at least some of the referral message.

28. A GUI according to claim 1, further comprising searching means for searching an externally accessible knowledge base, the searching means being arranged to convert a selected information topic into a predetermined classification code representing that topic and to transmit that classification code within an information request to the knowledge base for relevant information contained therein.

29. A GUI according to claim 28, wherein the classification code comprises a standard classification code describing a complete range of possible data inputs relevant to the subject of the workflow process.

30. A GUI according to claim 28, wherein the subject of the workflow process is clinical medical information and the classification code represents one of the group comprising a diagnosis, a symptom, an action, a treatment and an operative procedure.

31. A GUI according to claim 28, wherein the searching means is arranged to receive a response to the information request and display the results of the search to the user.

32. A GUI according to claim 28, wherein the searching means is arranged to receive a response to the information request and use the response to determine a relevant page of a plurality of pages for display to the user.

33. A GUI according to claim 28, wherein the searching means is arranged to display a plurality of information topics to the user and to enable selection of at least some of these information topics, each information topic being related to the current node location of the user within the current page..

34. A GUI according to claim 33, wherein the searching means is arranged to enable the user to enter additional information topics not displayed by the searching means.

35. A GUI according to claim 1, further comprising editing means for editing the plurality of interconnected nodes on a page, the editing means being arranged to update the stored workflow to reflect any change made to the page.

36. A GUI according to claim 35, wherein the editing means is arranged to enable a user to add a new node and to specify the contents of the new node.

37. A GUI according to claim 35, wherein the editing means is arranged to enable a user to specify functionality associated with a node.

38. A GUI according to claim 35, wherein the editing means is arranged to enable a user to add or edit a classification code associated with the contents of a node.

39. A GUI according to claim 35, wherein the editing means is arranged to enable user-controlled positioning of the new node within the page and interconnection of the new node to the existing plurality of interconnected nodes.

40. A GUI according to claim 1, further comprising recording means for recording user navigation through the plurality of interlinked nodes.

41. A GUI according to claim 40, further comprising navigation analysis means for analysing the user navigation to determine the precise path taken through the workflow process.

42. A GUI according to claim 41, wherein information relating to each step in the process is cost quantifiable and the navigation analysis means is arranged to determine a total cost of the path taken through the workflow process.

43. A GUI according to claim 41, wherein the navigation analysis means is arranged to analyse the performance of the user through the workflow process.

44. A graphical user interface (GUI) for interacting with a user during a workflow process, the GUI comprising:
a map comprising a plurality of interlinked nodes which graphically represent the structure of a plurality of interlinked steps of a stored workflow process;
a data entry module for entering data relating to a particular selected node; wherein the node has a unique relationship with a step in the workflow process;
a pathway module for determining a particular path through the workflow process using the entered data; and
a display module for graphically representing the resultant path through the workflow process in the map.

45. A graphical user interface (GUI) for providing a user interface to a knowledge base storing a workflow process, the GUI comprising:
a page including a plurality of interlinked nodes which graphically represent the structure of a plurality of interlinked steps of the stored workflow process;
means for entering data relating to a particular selected node; wherein the node has a unique relationship with a step in the stored workflow process;
means for determining a particular path through the workflow process using the entered data; and
means for graphically representing the resultant path through the workflow process in the page.

46. A graphical user interface (GUI) for interacting with a user during a workflow process, the GUI comprising:
a plurality of pages representing a plurality of interrelated workflows, each page comprising a plurality of interlinked nodes which graphically represent the structure of a plurality of interlinked steps within a stored workflow process;
data entry means for entering data relating to a particular selected node; wherein the node has a unique relationship with a step in the workflow process
determining means for determining a particular path through the workflow process using the entered data; and
graphical means for graphically representing the resultant path through the workflow process in the page.

47. A method of interacting with a user during a workflow process using a graphical user interface (GUI), the method comprising:
generating a page of the GUI, the page comprising a plurality of interlinked nodes which graphically represent the structure of a plurality of interlinked steps of the workflow process;
entering data relating to a particular selected node; the node having a unique relationship with a step in the workflow process;
determining a particular path through the workflow process using the entered data; and
graphically representing the resultant path through the workflow process in the page.

48. A graphical user interface (GUI) for interacting with a user during a workflow process, the GUI comprising:
searching means for searching an externally accessible knowledge base, the searching means comprising:
conversion means for converting a selected information topic into a predetermined classification code representing that topic; and
transmission means for transmitting that classification code within an information request over a communications network to the knowledge base to access relevant information contained therein.

49. A GUI according to claim 48, wherein the conversion means further comprises a local database of predetermined classification codes and an associated list of specific information topics which are each mapped to a specific classification code.

50. A GUI according to claim 48, wherein the classification code comprises a standard classification code describing a complete range of possible data inputs relevant to the subject of the workflow process.

51. A GUI according to claim 48, wherein the subject of the workflow process is clinical medical information and the classification code represents one of the group comprising a diagnosis, a symptom, an action, a treatment and an operative procedure.

52. A GUI according to claim 51, wherein the classification code comprises a SNOMED code.

53. A GUI according to claim 48, wherein the searching means is arranged to receive a response to the information request and display the results of the search to the user.

54. A GUI according to claim 48, wherein the searching means is arranged to receive a response to the information request and use the response to determine a relevant part of the workflow process to display to the user.

55. A GUI according to claim 48, wherein the searching means is arranged to display a plurality of information topics to the user and to enable selection of at least some of these information topics, each information topic being related to a current user accessed part of the workflow process.

56. A GUI according to claim 55, wherein the searching means is arranged to enable the user to enter additional information topics not displayed by the searching means.

57. A method of interacting with a user during a workflow process using a graphical user interface (GUI), the method comprising:
receiving a user instruction from the GUI to search an externally accessible knowledge base;
initiating a search of the knowledge base by:
converting a selected information topic into a predetermined classification code representing that topic; and transmitting that classification code within an information request over a communications network to the knowledge base to access relevant information contained therein.

58. A graphical user interface (GUI) for interacting with a user during a workflow process, the GUI comprising:
a page including a plurality of interlinked nodes which graphically represent the structure of a plurality of interlinked steps of a stored workflow process;
editing means for editing the plurality of interlinked nodes; and
updating means for updating the plurality of interlinked steps of the stored workflow process with any corresponding changes made to the plurality of interlinked nodes.

59. A GUI according to claim 58, wherein the editing means is arranged to enable a user to add a new node and to specify the contents of the new node.

60. A GUI according to claim 58, wherein the editing means is arranged to enable a user to specify functionality associated with a node.

61. A GUI according to claim 58, wherein the editing means is arranged to enable a user to add or edit a classification code associated with the contents of a node.

62. A GUI according to claim 58, wherein the editing means is arranged to enable user-controlled positioning of the new node within the page and interconnection of the new node to the existing plurality of interconnected nodes.

63. A GUI according to claim 58, wherein the subject of the workflow process is clinical medical information and the editing means is arranged to edit clinical information associated with a node.

64. A GUI according to claim 58, wherein the editing means is arranged to edit administration information associated with a node.

65. A system for supporting distributed interaction with a user during a workflow process, the system comprising:
a centrally stored graphical representation of the workflow process,
a plurality of users located remotely from the centrally stored representation and related to each other in a user hierarchy, each user having access to a version of the representation;
referral means provided within each version of the representation to generate a referral message, the referral means being arranged to send the message to a reviewer in a next higher level in the user hierarchy.

66. A system according to claim 65, wherein the referral means is arranged to receive a referral message from a user in a next lower level in the user hierarchy.

67. A system according to claim 65, wherein the referral means comprises forwarding means arranged to enable a reviewer to forward the message onto another reviewer at a next higher level within the hierarchy if required.

68. A system according to claim 65, wherein the referral means comprises response means enabling a reviewer to generate a message and send it to the referring user.

69. A system according to claim 68, wherein the response means is arranged to specify a response to the feedback message which can be used to update a monitoring function.

70. A system according to claim 69, wherein the monitoring function is accessible to all uses involved with the feedback message.

71. A system according to claim 69, wherein the response means is arranged to specify a resolution to the feedback message, which can be used to update the monitoring function.

72. A system according to claim 65, wherein the representation comprises a plurality of interlinked nodes which graphically represent the structure of a plurality of interlinked steps of the stored workflow process;

73. A system according to claim 72, wherein the referral means is provided at a node of the representation and using information associated with the node to populate at least some of the referral message on user-selection.

74. A system according to claim 65, wherein the referral means comprises a graphical icon and user selection comprises interaction between a user navigational tool and the icon.

75. A system according to claim 65, wherein the referral means is arranged to use information obtained from an electronic patient record to populate automatically at least some of the referral message.

76. A system for distributing a new version of a graphical user interface (GUI) to a user, the system comprising:
a central store retaining a GUI representation of a workflow process;
a plurality of users located remotely from the central store and related to each other in a user hierarchy below the central store, each user having access to a version of a previous representation;
comparing means for comparing the new version of the representation with a user's previous version of the representation to determine any differences;
forwarding means for forwarding those differences to the user associated with that version of the representation for consideration; and
reviewing means provided within each previous version of the representation, the reviewing means being arranged to accept or reject the differences and to convey an acceptance or rejection to a higher level within the hierarchy.

77. A system according to claim 76, wherein the reviewing means is arranged to accept some of the differences and to communicate the acceptance in part to a higher level within the hierarchy.

78. A system according to claim 76, wherein the reviewing means is arranged to enable the user to carry out the acceptance in part of the differences manually.

79. A system according to claim 76, wherein the GUI representation comprises a plurality of interlinked nodes which graphically represent the structure of a plurality of interlinked steps of the stored workflow process.

80. A system according to claim 76, wherein each user has an associated permission which determines the degree of changes that can be accepted at their particular level in the hierarchy.

81. A system according to claim 76, further comprising means for notifying each user of their position within the hierarchy and the permissions associated therewith.

82. A method of constructing a graphical user interface, the method comprising:
collating content regarding a particular workflow,
recording that content in a database as a series of steps of a hierarchically structured workflow, and
generating a graphical representation of the hierarchical workflow structure, which can be used to guide a user through the workflow; the graphical representation comprising a plurality of interlinked nodes where each node corresponds to a specific point within the hierarchical workflow structure.

83. A method according to claim 82, wherein the generating step comprises generating a graphical representation comprising the plurality of interlinked nodes on a single page.

84. A method according to claim 82, wherein the recording step comprises creating a workflow which commences with a fact in relation to one of a plurality of causes of the fact and the workflow steps provide a methodology to determine which of the plurality of causes is responsible for generating this fact.

85. A method according to claim 82, wherein the workflow is a clinical diagnosis workflow, and the fact comprises a symptom and the cause of the fact comprises a medical condition.
